(19) United States
(12) Patent Application Publication
(10) Pub. No.: US 2024/0148281 A1
(43) Pub. Date: May 9, 2024

(54) COLORIMETRIC SWEAT SENSING DEVICE AND METHODS OF MAKING THE SAME

(71) Applicant: THE COCA-COLA COMPANY, Atlanta, GA (US)

(72) Inventors: Timothy Charles KING, Harston (GB); Andrew Robert TAYLOR, Harston (GB); Rachel Elizabeth SMITH, Epsom (GB); Thomas James O’NEILL, Harston (GB); Laura Mae DAGLISH, Harston (GB); David John NIGHTINGALE, Harston (GB); Jack Lawrence WARREN, Harston (GB)

(21) Appl. No.: 18/272,891

(22) PCT Filed: Jan. 19, 2022
(86) PCT No.: PCT/US2022/012871

§ 371 Date: Jul. 18, 2023

Related U.S. Application Data
(60) Provisional application No. 63/138,964, filed on Jan. 19, 2021.
(60) Provisional application No. 63/276,860, filed on Nov. 8, 2021.

(51) Int. Cl. A61B 5/145; A61B 5/02; G01N 21/78

(52) U.S. Cl. CPC A61B 5/14517; A61B 5/02; G01N 21/78

(57) ABSTRACT

Disclosed herein are disposable devices for measuring sweat having low dead volumes and allowing a quick sensory response. Also disclosed herein are methods of making and using such devices.

### CROSS-REFERENCE TO RELATED APPLICATIONS

This application claims the benefit of U.S. Provisional Application No. 63/138,964 filed Jan. 19, 2021, and U.S. Provisional Application No. 63/276,860 filed Nov. 8, 2021, the contents of which are incorporated herein by reference in their entirety.

### TECHNICAL FIELD

The present invention generally relates to devices configured to measure and monitor sweat and methods of making the same. The present disclosure also relates to methods of measuring sweat using the disclosed herein sweat sensing devices.

### BACKGROUND

It is often advantageous to monitor and carry out an analysis of both the production rate and the composition of a person's sweat while they are performing strenuous physical exertion since such knowledge enables the individual to more accurately replenish lost sweat fluid and electrolytes.

However, sweat volume and sweat electrolytes vary in composition between individuals, and for a given individual, the sweat volume can depend on a sweating rate, which is itself a function of temperature, humidity, exertion rate, etc. Sports fluid replacement drinks help maintain electrolyte balance, but the optimum electrolyte level for a given individual in their current state is also variable. Too high or too low electrolyte levels can hurt taste perception, as well as negatively affect the body, such as high blood pressure or hyponatremia, and the individual needs to be educated to select the optimum beverage to maintain their physiology in the optimum state.

Thus, it is important to enable an individual to determine their electrolyte loss by sweating and so help them replenish their electrolytes optimally, enabling further exertion to be maximized. This knowledge would help support better competitive performance and also more effective training regimes.

Often, single-use, disposable analysis devices are preferred to determine electrolyte levels as, if used as intended, they are not susceptible to contamination from previous use.

Accordingly, a need exists for low-cost devices capable of accurately measuring sweat electrolytes in small volumes without interference from the skin. There is also a need for devices that are free of expensive components and simple for use and manufacture. These needs and other needs are at least partially satisfied by the present disclosure.

### SUMMARY

The present invention is directed to a device comprising: a) a substrate configured to be removably attached to a subject's skin, wherein the substrate is in fluid communication with at least the subject's skin and configured to receive and transfer a sweat aliquot; b) at least one sweat collector element disposed on the substrate such that it is in fluid communication with the substrate and has at least one inlet configured to receive the sweat aliquot; wherein the at least one sweat collector element has a fluidic configuration such that a flow of the sweat aliquot is directed along a longitudinal axis and/or an axial axis of the at least one sweat collector element; c) at least one sensor portion that is in fluid communication with the at least one sweat collector element; wherein the at least one sensor portion is configured to quantitively detect at least one characteristic of the sweat; and wherein the device is configured to indicate a presence of the at least one characteristic of the sweat.

The present invention is further directed to a device comprising a) a substrate configured to be removably attached to a subject's skin, wherein the substrate has at least one inlet in fluid communication with at least the subject's skin and wherein the at least one inlet is configured to receive and transfer a sweat aliquot; b) at least one sweat collector element configured to receive the sweat aliquot from the at least one inlet of the substrate; c) at least one sensor element having a proximal end and a distal end, a first length, l₁, along a longitudinal axis of the device, measured between the proximal end and the distal end of the at least one sensor element, wherein the at least one sensor element is in fluid communication with the at least one sweat collector element; and wherein the at least one sensor element is configured to quantitatively detect at least one characteristic of the sweat; d) at least one ready indicator element that is in fluid communication with the at least one sweat collector element through at least one fluidic path having a proximal end and a distal end and having a second length, l₂, as measured between the proximal and distal ends along the longitudinal axis of the device, wherein the second length is greater than the first length (l₂>l₁); and wherein the device is configured to indicate a presence of the at least one characteristic of the sweat.

In some exemplary aspects, the at least one sensor portion disclosed herein is in intimate contact with the sweat collector element. Also disclosed are aspects where the sensor portion comprises a colorimetric reagent configured to react with the at least one characteristic of the sweat.

In still further aspects, the at least one characteristic of the sweat comprises an amount of cations, anions, pH, temperature, volume, an amount of biomarkers, or a combination thereof. In still further aspects, the biomarkers can comprise glucose, cholesterol, uric acid, urea, ascorbic acid, lactate, electrolytes, proinflammatory cytokines, hormones, tuberculosis-specific proteins, Parkinson-specific proteins, schizophrenia-specific proteins, cancer-specific metabolic markers, cystic fibrosis specific markers, drug-specific markers, alcohol, or any combination thereof.

Also disclosed herein is a device comprising: a) a substrate that is configured to receive and transfer a fluid aliquot; b) at least one fluid collector element disposed on the substrate such that it is in fluid communication with the substrate and has at least one inlet configured to receive the fluid aliquot; wherein the at least one fluid collector element has a fluidic configuration such that a flow of the fluid aliquot is directed along a longitudinal axis and/or an axial axis of the at least one fluid collector element; c) at least one sensor portion that is in fluid communication with the at least one fluid collector element; wherein the at least one sensor portion is configured to quantitively detect at least one characteristic of the fluid; and wherein the device is configured to indicate an amount of the at least one characteristic of the fluid, wherein the fluid is a biofluid or a non-biofluid.

Also disclosed herein is a device comprising: a) a substrate having at least one inlet that is configured to receive and transfer a fluid aliquot; b) at least one collector element configured to receive the fluid aliquot from the at least one inlet of the substrate; c) at least one sensor element having a proximal end and a distal end, a first length, l₁, along a longitudinal axis of the device, measured between the proximal end and the distal end of the at least one sensor element, wherein the sensor element is in fluid communication with the at least one collector; and wherein the at least one sensor element is configured to quantitatively detect at least one characteristic of the fluid; d) at least one ready indicator element that is in fluid communication with the at least one collector through at least one fluidic path having a proximal end and a distal end and having a second length, l₂, as measured between the proximal and distal ends along the longitudinal axis of the device, wherein the second length is greater than the first length (l₂>l₁); and wherein the device is configured to indicate a presence of the at least one characteristic of the fluid, wherein the fluid is a biofluid or a non-biofluid.

In such exemplary aspects, the biofluid can comprise sweat, saliva, blood, urine, tears, reproductive fluids, or any combination thereof.

Additional aspects of the disclosure will be set forth, in part, in the detailed description, figures, and claims which follow, and in part will be derived from the detailed description, or can be learned by practice of the invention. It is understood that both the foregoing general description and the following detailed description are exemplary and explanatory only and are not restrictive of the invention as disclosed.

### BRIEF DESCRIPTION OF DRAWINGS

FIG. 1 depicts a schematic of an exemplary device applied to the skin in one aspect.

FIG. 2 depicts a top layer of an exemplary device in one aspect.

FIG. 3 depicts an exemplary assembly configured to test an exemplary device, as disclosed in the aspects described herein.

FIG. 4 depicts a visual measurement of at least one sweat property as measured by an exemplary device schematically shown in FIG. 1.

FIG. 5 depicts a schematic of an exemplary device applied to the skin in one aspect.

FIGS. 6A-6E show individual components of the exemplary device shown in FIG. 5.

FIG. 7 depicts a visual measurement of at least one sweat property as measured by the exemplary device schematically shown in FIG. 5.

FIG. 8 depicts a number of bars present in the exemplary device shown in FIG. 1 that show a visual measurement as a function of sodium chloride concentration.

FIG. 9 depicts a correlation between a degree of color change and sweat analyte concentration as measured by the exemplary device schematically shown in FIG. 5.

FIG. 10 depicts a schematic of an exemplary device applied to the skin in a different aspect.

FIG. 11 depicts individual components of the exemplary device shown in FIG. 10.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the singular forms “a,” “an,” and “the” include plural referents unless the context clearly dictates otherwise. Thus, for example, reference to a “device” or an “electrode” includes aspects having two or more such devices or electrodes unless the context clearly indicates otherwise.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used herein, the terms “optional” or “optionally” mean that the subsequently described event or circumstance may or may not occur, and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein interpreted accordingly.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one embodiment to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, characteristic, component, composition, or other condition for which substantially is used to characterize or otherwise quantify an amount.

In other aspects, as used herein, the term “substantially free,” when used in the context of clogging of a channel, for example, is intended to refer to a channel that is less than about 5% clogged, less than about 4% clogged, less than about 3 of clogged, less than about 2% clogged, less than about 1% clogged, less than about 0.5% clogged, less than about 0.1% clogged, or less than about 0.01% clogged.

In other aspects, as used herein, the term “substantially no,” when used in the content of contact of a disclosed surface with any other surface, for example, is intended to refer to that the disclosed surface does not contact any other surface or it contacts in a manner that does affect any properties of either surface or device.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “sweat” refers to a biofluid that is primarily sweat, such as eccrine or apocrine sweat, and may also include mixtures of biofluids such as sweat and blood, or sweat and interstitial fluid, or sweat and any other fluid that can be found in its vicinity, so long as advective transport of the biofluid mixtures (e.g., flow) is primarily driven by sweat.

As used herein, the term “measured” can refer in some aspects an exact or precise quantitative measurement, while in other aspects, it can also refer to measuring relative amounts, rates of change, or qualitative data. It is understood that any value that is measured can be presented in any form. In certain aspects, the data can be presented as a final concentration, as a range, as a qualitative response of “yes” or “no,” or any other form that conveys any sought information.

Similarly, when the disclosure refers to a device configured to indicate a presence of the at least one characteristic of the fluid (or sweat), it is understood that the term “presence” can include numerical values, visual representation, qualitative response, etc. It is further understood that this term, as used in the specified context, can also include a simple presence of the indicated property, relative amounts of the indicated property, ranges of various amounts, calibrated amounts against different reagents or components, or even precise amounts when possible. In yet further aspects, the term “presence” can indicate ranges such as, for example, “low,” “medium,” and/or “high.” It can also indicate any ranges in between. This indication can be done visually by the color scale, or it can also show specific wording or number as desired. It is further understood that any representation that helps the device's wearer estimate the amount of the indicated property is included in this disclosure.

Numerous other general purpose or special purpose computing devices environments or configurations can be used. Examples of well-known computing devices, environments, and/or configurations that can be suitable for use include, but are not limited to, personal computers, server computers, handheld or laptop devices, smartphones, multiprocessor systems, microprocessor-based systems, network personal computers (PCs), minicomputers, mainframe computers, embedded systems, distributed computing environments that include any of the above systems or devices, and the like.

Computing devices, as disclosed herein, can contain communication connection(s) that allow the device to communicate with other devices if desired. Computing devices can also have input device(s) such as a keyboard, mouse, pen, voice input device, touch input device, etc. Output device(s) such as a display, speakers, printer, etc., can also be included. All these devices are well known in the art and need not be discussed at length here.

Computer-executable instructions, such as program modules being executed by a computer, can be used. Generally, program modules include routines, programs, objects, components, data structures, etc., that perform particular tasks or implement particular abstract data types. Distributed computing environments can be used where tasks are performed by remote processing devices that are linked through a communications network or other data transmission medium. In a distributed computing environment, program modules and other data can be located in both local and remote computer storage media, including memory storage devices.

In its most basic configuration, a computing device typically includes at least one processing unit and memory. Depending on the exact configuration and type of computing device, memory can be volatile (such as random-access memory (RAM)), non-volatile (such as read-only memory (ROM), flash memory, etc.), or some combination of the two.

Computing devices can have additional features/functionality. For example, a computing device can include additional storage (removable and/or non-removable), including, but not limited to, magnetic or optical disks or tape.

Computing device typically includes a variety of computer-readable media. Computer-readable media can be any available media that can be accessed by the device and includes both volatile and non-volatile media, removable and non-removable media.

Computer storage media include volatile and non-volatile, and removable and non-removable media implemented in any method or technology for storage of information such as computer-readable instructions, data structures, program modules, or other data. Memory, removable storage, and non-removable storage are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device. Any such computer storage media can be part of a computing device.

Computing devices, as disclosed herein, can contain communication connection(s) that allow the device to communicate with other devices. The connection can be wireless or wired. Computing devices can also have input device(s) such as a keyboard, mouse, pen, voice input device, touch input device, etc. Output device(s) such as a display, speakers, printer, etc., can also be included. All these devices are well known in the art and need not be discussed at length here.

It should be understood that the various techniques described herein can be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, can take the form of program code (i.e., instructions) embodied in tangible media, such as CD-ROMs, hard drives, or any other machine-readable storage medium where, when the program code is loaded into and executed by a machine, such as a computer, the machine becomes an apparatus for practicing the presently disclosed subject matter.

Also, it is further understood that the devices disclosed herein can be in communication with other computerized devices by various means. In certain aspects, the sensors can transfer the information to the computerized devices with cameras or any other devices configured to capture a visual response of the measuring device. In yet other aspects, where the measuring device response is a change of color, it is understood that the change of color can occur in visual spectra of the light.

It is further understood, however, that the measuring response device can also occur in UV or IR spectra. In such aspects, the response can be further evaluated by additional means and the final result presented to the device wearer. It is also understood that the response of the devices disclosed herein can include photo fluorescence, fluorescence, and/or luminescent responses.

While aspects of the present invention can be described and claimed in a particular statutory class, such as the system statutory class, this is for convenience only and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Moreover, for the sake of simplicity, the attached figures cannot show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present invention may be understood more readily by reference to the following detailed description of various aspects of the invention and the examples included therein and to the Figures and their previous and following description.

### Device

In some aspects, disclosed herein is a device configured to collect sweat from a surface on a subject's skin and guide it to a sensing area. It is understood that the devices disclosed herein can be placed anywhere on the skin. For example, and without limitations, it can be placed on the upper arm, forearm, thigh, calf, or any other area of skin that has eccrine sweat glands. In still further aspects, the mechanical properties of the device are such that it can substantially conform to the shape of the body part it has been attached to.

The disclosed device comprises a substrate, at least one sweat collector element, and at least one sensor portion. In certain aspects, the disclosed substrate is configured to be removably attached and be in fluid communication with at least the subject's skin and configured to receive and transfer a sweat aliquot.

In still further aspects, the at least one sweat collector element can be disposed on the substrate such that it is in fluid communication with the substrate. The sweat collector element also has at least one inlet that is configured to receive the sweat aliquot.

In yet still further aspects, the at least one sensor portion is in fluid communication with the at least one sweat collector element, wherein the at least one sensor portion is configured to quantitively detect at least one characteristic of the sweat.

Exemplary and unlimiting devices are shown in FIGS. 1, 5, and 10, each of which is described in detail below.

As shown in FIG. 1, the device can have, for example, a rectangular form. It is understood, however, that this form is exemplary and unlimiting, and any other possible configurations can be utilized. In these exemplary aspects, the device is a rectangular strip having any desired dimensions. In certain aspects, the device is sized to minimize the volume of sweat that is required to be collected and to enable a reduction of the time to saturation and thus to minimize the time for receiving the measurement result. It is understood, however, the smallest dimension of the device will be limited by the ability of the wearer to determine the measurement results. Yet, in other aspects, it is also understood that having large devices can be inconvenient to the wearer and not economically beneficial. Also, it is understood that the larger device can require a longer time for the sweat collection and, as a result, a longer time for receiving the results that can also be imprecise due to undesirable sweat evaporation.

In still further aspects, the size of the exemplary device can be anywhere between about 30 mm to about 70 mm in length, including exemplary values of about 35 mm, about 40 mm, about 45 mm, about 50 mm, about 55 mm, about 60 mm, and about 65 mm in length. Yet in other aspects, the size of the exemplary device can be anywhere between about 1 mm to about 15 mm in width, including exemplary values of about 1.5 mm, about 2 mm, about 2.5 mm, about 3 mm, about 3.5 mm, about 4 mm, about 4.5 mm, about 5.5 mm, about 6 mm, about 6.5 mm in width, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, and about 14.5 mm.

In yet other aspects, it is understood that the sensor portion can occupy all the width and/or length of the device, or it can occupy a portion of the device. For example, the sensor portion can have any width between 1 mm to about 5 mm, including exemplary values of about 1.5 mm, about 2 mm, about 2.5 mm, about 3 mm, about 3.5 mm, about 4 mm, about 4.5 mm.

In still further aspects, an overall thickness of the device can be anywhere between 80 μm to about 200 μm, including exemplary values of about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, and about 195 μm.

In still further aspects, and as disclosed above, the device can have any shape. It does not have to be rectangular; it can be square, rhombic, triangular, irregular shape, trapezoid shape, or any other shape that can provide a flow path such that sweat (or any other fluid if desired) aliquot can enter at one end and optionally exits at the other, passing through the device on the way. Yet, in other aspects, the aliquot can saturate the device's components without exiting the device. The devices described herein can comprise multiple stacked layers to add additional functionalities or protect components and reduce the risk of extraneous environmental fluids entering the system. The devices may have a tapered geometry that increases the adhesion to the skin, promotes the formation of a seal to prevent extraneous liquid from reaching the inlet, and reduces the risk of delamination. The described devices can be flexible, stretchable, and can establish conformal contact with the underlying skin surface.

Further referring to FIG. 1, a device 100 includes a substrate 102, a sweat collection layer 104, and a sensor portion 106. The substrate 102 has a proximal portion and a distal portion and an inlet 102c that is configured to receive and transfer the sweat aliquot. The inlet of the substrate is disposed in the proximal portion of the substrate. It is understood that the terms “proximal” and “distal” as used herein are relative terms to describe each of the sensors' terminals relative to each other. The substrate can be any substrate that is inexpensive, easy to produce, flexible, and can be easily adhered to the skin of the wearer without causing any discomfort. In some exemplary and unlimiting aspects, the substrate can comprise silicone, polyurethane, polyvinyl chloride (PVC), polyethylene terephthalate (PET), thermoplastic elastomers, nylons, metalized-PET, waxed papers or modified waxed papers, polydimethylsiloxane (PDMS), cellulose paper, cellulose sponge, polyurethane sponge, polyvinyl alcohol sponge, silicone sponge, polystyrene, polyimide, SU-8, wax, olefin copolymer, polymethyl methacrylate (PM MA), polycarbonate, poly(styrene-isoprene-styrene), chitosan, and any combination thereof. In certain aspects, the substrate comprises any materials that are non-absorbing and have a substantially moderate-to-low water vapor transfer rate.

In still further aspects, the substrate can also comprise adhesive layers, for example, the adhesive layer 102a that assist in adhering the substrate to the wearer's skin. Any known in the art adhesive layers can be utilized. However, it is understood that adhesives that are biocompatible, non-allergenic, non-irritating are preferred.

In some aspects, the adhesives can be pressure adhesives, for example, pressure-sensitive adhesive. Yet, in other aspects, the adhesives can comprise soft silicone adhesive. Some exemplary and unlimiting examples of the adhesive layers include a double-sided 3M 2477P, single-sided 3M 2475P, single-sided Vancive 5500SI.

In still further aspects, a surface of the substrate that is opposite from a surface of the substrate that is in contact with the wearer's skin (e.g., opposite from the adhesive layer 102a) can further comprise one or more impermeable layers 102b. Such layers again can be selected from silicone, polyurethane, polyvinyl chloride (PVC), polyethylene terephthalate (PET), thermoplastic elastomers, nylons, metalized-PET, waxed papers or modified waxed papers, polydimethylsiloxane (PDMS), cellulose paper, cellulose sponge, polyurethane sponge, polyvinyl alcohol sponge, silicone sponge, polystyrene, polyimide, SU-8, wax, olefin copolymer, polymethyl methacrylate (PM MA), polycarbonate, poly(styrene-isoprene-styrene), chitosan, and any combination thereof.

The inlet 102c is disposed in the proximal portion of the substrate to allow the sweat aliquot to enter the device 100.

The sweat collection layer 104 is shown in FIG. 1 and comprises a sweat collector element 104a, a sweat sensing component 104b, and a ready indicator 108. It is understood that in some aspects, more than one sweat collector element 104a can be present. In certain aspects, at least one sweat collector element 104a has a proximal portion and an opposite distal portion, wherein the at least one inlet of the sweat collection layer 104 is disposed at the proximal portion of the at least one sweat collector element 104a. In further aspects, the inlet of the at least one sweat collection layer 104 and the inlet of the substrate 102c are substantially aligned. In still further aspects, the flow of the sweat aliquot can be gradual and longitudinal from the proximal portion to the distal portion of the sweat collector element 104a. The at least one sweat collector element 104a, as shown in FIG. 1, can comprise a wicking element for directing a flow of the sweat aliquot along the sweat collector element 104a that is disposed substantially close to and in fluid communication with the substrate inlet 102c and at least one sensor portion 104b disposed within the sweat collection layer 104. It is understood that in this specific and unlimiting example, the sweat collector element 104a and the at least one sensor portion 104b are disposed within the same sweat collection layer 104. However, different configurations are also contemplated and can be determined by the specific application.

In certain aspects, the sweat collector element 104a can comprise any material configured to collect the sweat or any other fluid. In still further aspects, a fluidic connection between various portions of the device is such that the sweat that has entered at a proximal end of the element cannot reach the distal end of the element without first passing through the rest of the element.

In still further aspects, the sweat collector element 104a can comprise a porous material configured to directionally adsorb and transfer the sweat aliquot. In some exemplary and unlimiting aspects, the porous material can comprise one or more layers. For example, the porous material can comprise two or more layers. In still further aspects, the sweat collector element 104a can comprise a paper, a polymer, a textile, a foam, or any configuration thereof. In further examples, and without limitations, the sweat collecting element can comprise a paper or any other cellulosic material. In certain aspects, the sweat adsorption is capillary. It is also understood that the exemplary sweat collector element materials disclosed herein can also be used in other devices described in this application.

It is further understood that the sweat collector element 104a can have any desired shape. In some aspects, the sweat collector element 104a can have a shape similar to the shape of the substrate. However, it is understood that the shape of the sweat collector element 104a does not have to be the same as the substrate. It is further understood that from convenience, manufacturing, and possible esthetics, the sweat collector element 104a, and in fact, all other elements disclosed below, have dimensions that are substantially similar or smaller than those of the substrate. In such aspects, all other components of the device are disposed within the substrate dimensions. The features disclosed herein also apply to other devices described in this specification. The geometry of each layer can be chosen independently of other layers to conform to the desired design.

In some aspects, the sweat collector element 104a can have a rectangular shape. But the sweat collector element 104a can have any other shape, regular or irregular.

The device 100 further comprises at least one sensor portion, as shown for example, and without limitations in FIG. 1 as a component 104b. In some aspects, the at least one sensor portion 104b is in intimate contact with the sweat collector element 104a. In some exemplary and unlimiting aspects, the at least one sensor portion 104b can be at least partially embedded within the sweat collector element 104a. While in other aspects, the at least one sensor portion 104b can be disposed as a separate layer that is in intimate contact with the sweat collector element 104a.

It is further understood, however, that more than one sensor portion 104b can be present. In some aspects, one sensor portion such as, for example, 104b can be at least partially embedded within the sweat collector element 104a, while a second sensor portion (not shown) can be disposed in a separate layer that is in intimate contact with the sweat collector element 104a.

In some aspects, the at least one sensor portion 104b can be a continuation of the sweat collector element 104a. While in other aspects, the at least one sensor portion (not shown) can be a separate element that is either embedded with the sweat collector element 104a or is in substantially intimate contact with the sweat collector element 104a.

As shown in FIG. 1, for example, and without limitations, the sensor portion 104b can be embedded along a length of the sweat collector element 104a between the proximal portion and the distal portion of the sweat collector element 104a. While in yet further aspects, a separate layer with the sensor portion 104b can be disposed along a length of the sweat collector element 104a between the proximal portion and the distal portion of the sweat collector element 104a.

In still further aspects, the sensor portion 104b can comprise a reagent configured to react with the at least one characteristic of the sweat, thereby alerting the wearer to the specific amount of this at least one characteristic. In some aspects, the reagent is a colorimetric reagent. However, it is also understood that the reagent can be any reagent that can react with the at least one characteristic of the sweat to produce a product or a different reagent that can be correlated with the presence of the at least one characteristic. For example, and without limitation, the reaction between the reagent and the at least one characteristic of the sweat can result in a fluorescent, photo fluorescent, or luminescent signal. It can also result in a response that can be measured in the UV or IR spectrum of the light. Yet, in other aspects, when the colorimetric reagent is used, it shows a change in color in a visible spectrum providing the device's wearer with quick identification of the measurement.

In still further aspects, any known in the art colorimetric reagents can be sued. For example, and without limitation, the colorimetric reagent can comprise silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion or iron ion, a 2,2-bicinchoninic acid, a, 10-phenanthroline, a universal pH indicator, silver dichromate, a compound of fluorescein and silver nitrate, or any combination thereof. In still further aspects, the colorimetric reagent is 2,4,6-tris(2-pyridiyl)-s-triazine (TPTZ) complexed with mercury ion, silver ion, or iron ion; or silver chloroanilate. In still further exemplary and unlimiting examples, the at least one sensor portion of the devices disclosed herein, such as, for example, 104b, can comprise 4,6-tris(2-pyridiyl)-s-triazine (TPTZ) complexed with mercury ion, silver ion, or iron ion; or silver chloroanilate.

In still further aspects, the colorimetric reagent can be present in an amount effective to determine a dynamic range of a concentration of the at least one characteristic of the sweat. While in other aspects, the colorimetric reagent can be present in an effective composition to determine a dynamic range of a concentration of the at least one characteristic of the sweat. In such exemplary aspects, the reagent can comprise various compositions and include more than one component, and therefore the dynamic range of the concentration of the at least one characteristic of the sweat can also be dependent on a ratio of these components within the reagent.

It is understood that the concentration of the reagent can be the same or different throughout the sensor portion 104b. In some aspects, the amount of the colorimetric reagent can be substantially similar per unit area. While in other aspects, the sensor portion 104b can have a plurality of portions having different concentrations of the reagent.

It is understood that the specific chemistry of the reagent in the sensor portion 104b can be selected such that the length of the element that changes color can show the most suitable dynamic range for the purpose (i.e., no color change occurs at very low analyte concentrations while the entire length of the element changes color at the highest expected analyte concentrations). In such exemplary aspects, the at least one characteristic of the sweat is exposed to an increasing cumulative amount of reagent as the sweat aliquot proceeds from the proximal portion of the sweat collector element 104a to the distal portion of the sweat collector element 104a. In yet other aspects, the sensor portion 104b is configured to indicate a concentration of the at least one characteristic of the sweat by a substantial change of color of the colorimetric reagent from the proximal portion to the distal portion of the sweat collector element 104a. In some aspects, the change of color is substantially linear. While in other aspects, the change of color is linear. While in still further aspects, the change of color is substantially non-linear. In yet further aspects, the change of color is at least partially linear or partially non-linear. In still further aspects, the change of color can achieve saturation.

It is understood that the mechanism of the reaction between the reagent and the at least one characteristic of the sweat is dependent on the reagent and the specific characteristic. It is further understood that the reagent can be disposed along a length of the sensor portion. As discussed in detail herein, the reagent can have the same concentration per unit area of the sensor portion or different depending on the desired application. In certain aspects, the sweat aliquot can travel along the portion 104b such that the at least one characteristic of the sweat reacts with the reagent present in the sensor portion 104b at its first point of contact, removing at least a portion of the at least one characteristic of the sweat from the sweat. As more sweat enters the sensor portion 104b, it needs to travel further along the sensor portion 104b before it encounters an un-reacted reagent left in the sensor portion 104b that can react with at least one characteristic of the sweat in the new aliquot causing a change in color. Again, as discussed above, the change can be substantially linear, partially linear, substantially non-linear, or partially non-linear.

In certain aspects, the sensor portion 104b, as disclosed above, can comprise two or more sensor portions. For example, and without limitations, multiple elements with different concentrations of the reagent can be used to increase the accuracy and precision of readings. However, it is also understood that consideration needs to be given to a potential increase in the volume of sweat required to be absorbed by the device.

An occlusive impermeable layer 110 can then be disposed on the sweat collection layer 104, the device 100. It is understood that these impermeable layers can prevent the reagent, the sweat aliquot, or a reaction product between the reagent and at least one characteristic of the sweat aliquot to evaporate or in any other way to affect the measurement reading. The impermeable layer 110 has an adhesive layer 110a disposed on a bottom portion of the impermeable layer that assists in adhering the impermeable layer to the underlying layers of the device 100 and an impermeable polymer layer 110b disposed on a top portion. Any of the impermeable polymers or materials disclosed herein can be used for this purpose.

As shown in FIG. 1, the device 100 can comprise an asymmetric indicator membrane 106. The asymmetric indicator membrane 106 is also in fluid communication with the at least one sweat collector element 104a. In such exemplary aspects, the asymmetric indicator membrane 106 can be any material having an axial gradient. For example and without limitations, the asymmetric indicator membrane 106 can be a porous membrane having an axial gradient in pore size, wherein the bottom portion of the asymmetric indicator membrane has a larger pore size than an upper portion, and wherein the upper portion of the asymmetric indicator membrane comprises a water-soluble dye configured to be dissolved in the sweat aliquot. It is understood that any water-soluble dies can be used. For example, and without limitations, Brilliant Blue FCF can be utilized. In still further aspects, an additional asymmetric porous membrane 108 is positioned above the asymmetric indicator membrane 106. In still further aspects, the dye can be applied to the side of the asymmetric membrane 106 with the smaller pore size in order to constrain the position of the dye due to the preferential wicking of the membrane.

The device 100 can also comprise a patterned upper surface or indicator layer 112. The indicator layer 112 is shown in more detail in FIG. 2. It is understood that this layer is optional. In some aspects, this layer can be helpful to the device wearer to read and understand the measurement results more clearly. In such aspects, the indicator layer can also show when the device is ready for reading. In such exemplary aspects, the indicator layer together with the asymmetric membrane 106 and asymmetric porous membrane 108 demonstrate that all reagents have reacted with the sweat aliquot, and the device is ready for reading.

In some aspects, the indicator layer 112 can be disposed on the sweat sensing element 104b and configured to visually segment the presence of the at least one characteristic of the sweat. In such aspects, as shown in FIG. 2, the indicator layer can have, for example, a window 111 to view the ready indicator membrane 106 when it changes its color to indicate that the device 100 is ready to be read. The indicator layer can also have divisions 113 of segments of the sweat sensing element 104b to view when the colorimetric reagent has reacted with the at least one characteristic of the sweat. Upon indication by the ready indicator 106 (with or without porous membrane 108), a user may see a number of divisions 113 which have undergone a color change in order to measure an amount of the characteristic of the sweat lost via sweat. It is understood that the indicator layer can be used to indicate which color the element should start and/or finish to assist users in determining if a portion of the element has or hasn't changed color. It is understood that the indicator layer shown in FIG. 2 is only exemplary. It can have any shape, structure, or design. For example, the windows segments of the divisions 113 that correlate to a specific amount of the measured characteristic can be represented by a logo or by a specific phrase, or dots, or stars, or any other shapes. For example, and without limitation, the segments can spell a word “DRINK,” a word “READY,” “EXPIRED,” “WARNING,” “ALERT,” or any other phrases or words that would bring the device wearer's attention to take or not to take a specific action.

The devices disclosed herein can measure any characteristics of the sweat. In some aspects, the at least one characteristic of the sweat can comprise an amount of cations, anions, pH, temperature, volume, an amount of biomarkers, or a combination thereof. Even further, the at least one characteristic of the sweat can be, for example, the amount of cations selected from sodium, potassium, calcium, magnesium, aluminum, iron, or a combination thereof. In yet still further aspects, the at least one characteristic of the sweat is the amount of anions selected from chlorides, lactates, or a combination thereof.

In some aspects, the at least one characteristic of the sweat can be present in an amount from about 0.10 mM to about 500 mM, including exemplary values of about 0.2 mM, about 0.5 mM, about 1 mM, about 5 mM, about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, and about 475 mM.

Again as disclosed above, any known in the art colorimetric reagents can be used. For example, and without limitation, the colorimetric reagent can comprise silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion or iron ion, a 2,2-bicinchoninic acid, a, 10-phenanthroline, a universal pH indicator, silver dichromate, a compound of fluorescein and silver nitrate, or any combination thereof. In still further aspects, the colorimetric reagent is 2,4,6-tris(2-pyridiyl)-s-triazine (TPTZ) complexed with mercury ion, silver ion, or iron ion; or silver chloroanilate.

In still further aspects, the colorimetric reagent used in any of the devices disclosed herein can be impregnated within the at least one sensor portion (for example, within 104b for device 100) from a solution, a suspension, or formed in-situ by reaction with the at least one characteristic of the sweat, or by a printing method.

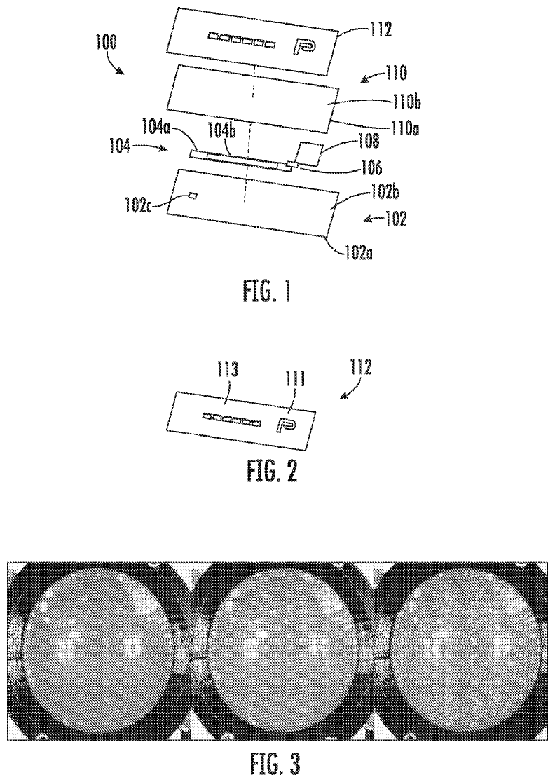

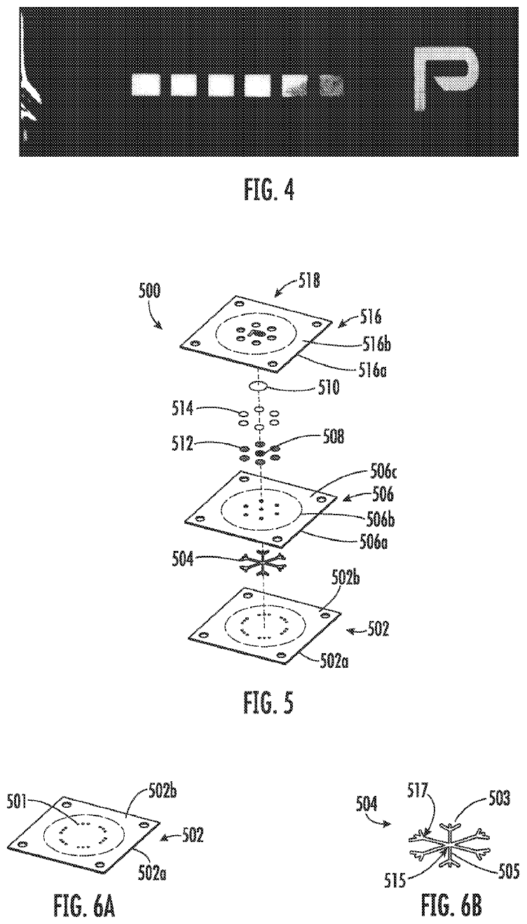

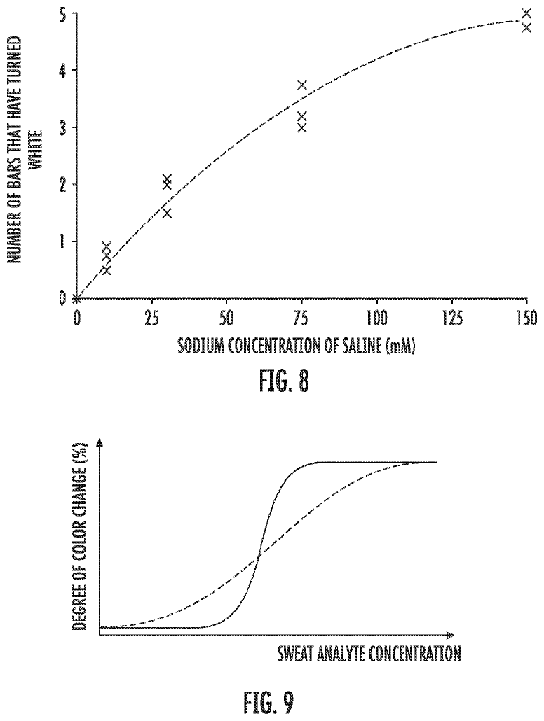

FIG. 3 shows an exemplary testing assembly used to imitate sweat production. A test assembly as shown in FIG. 3 was produced to replicate sweating skin by sealing a fluid-filled reservoir with two layers of silicone—a lower layer containing small pores (<1 μm) to control the flow rate and an upper layer containing an array of laser-cut holes (20-100 μm) to represent the size and spacing of sweat pores in human skin. The reservoir was gravity fed with a solution of deionized water and sodium chloride, resulting in a sweat rate comparable to the rate resulting from moderate to heavy exercise. The disclosed herein device was stuck onto this test rig and observed as it collected the solution. It was observed that the sweat was absorbed as anticipated, and the color change did correspond to the chosen concentration of sodium chloride within the solution. FIG. 4 shows a response of the device 100 during the described measurements. FIG. 8 shows a correlation of the distance (or a number of segments that have to change the color) vs. sodium ions concentration.

An additional aspect of the disclosed herein device is shown in FIGS. 5-7.

Now referring to FIG. 5. FIG. 5 shows a schematic of an exemplary multi-layer device 500 that comprises a substrate 502. The substrate 502 comprises an adhesive material 502a used to attach the device to the wearer's skin and an impermeable layer 502b disposed on a top surface of the device 500 opposite from a surface that is in intimate contact with the skin (e.g., opposite from the adhesion material 502a). Any of the disclosed herein substrates, adhesive materials, and impermeable polymers (layers) can be used in the construction of this exemplary device. It is understood, however, that in choosing the adhesive material, one should consider the convenience of the device's wearer. The device 500, as well as device 100, or any other device in the scope of this disclosure, should be easily attached and removed, stay in place during exercise and in the presence of sweat, not to cause skin irritation, etc.

In some exemplary and unlimiting aspects (not shown), the substrate 502 does not comprise an adhesive material 502a. It is understood that such aspects can exist for any other devices falling within the scope of this disclosure. For example, device 100 can also have a substrate 102 that does not comprise an adhesive material. In such aspects, the attachment can be done, for example, with an adhesive drape that lies over the top of the device and adheres to the skin around the device. However, such a design may result in the formation of the air pockets under the device 500 (or 100) that would require extra sweat to fill before the sweat is absorbed by the device 500 (or 100) and thus affecting the response time. In other aspects, the device 500 (or 100) can also be attached mechanically to the skin with some fasteners.

In still further aspects, any of the disclosed below layers of the multi-layer devices can have an appropriate thickness. It is understood that these aspects are relevant to devices 100 and 500 and can also be applied to any other devices within the scope of this disclosure. The thickness of each layer can vary. It is understood that the minimum and maximum available thicknesses can be determined by the desired properties, such as barrier properties for impermeable layers, wicking for porous layer, adhesion for the adhesive layer, etc., and by the desire to reduce the total dead volume in the patch.

In still further aspects, the multi-layer construction of the device 500 (or 100) disclosed herein allows the sweat aliquot to both laterally within a layer and vertically between absorbent layers.

Similarly, the device 500 itself, the substrate 502, and any subsequent layers can have any desired shape. For example, and without limitations, the shape can be square, rectangular, rhombic, trapezoid, triangular, star-shaped, or any other regular or irregular shape, depending on the desired application. The exemplary and unlimiting device shown in FIG. 5 can have a size of about 40 mm across. However, this size of the device is only exemplary, and specific sizing can be determined by one of the ordinary skills in the art. For example, the overall size of the device can be determined by the wearer's convenience. If the device is too small, the visualization of the results would be difficult for the device wearer. In other aspects, in choosing the device size, one should take into consideration an increase in the volume of sweat collection elements and a possible increase in the dead volume in the device.

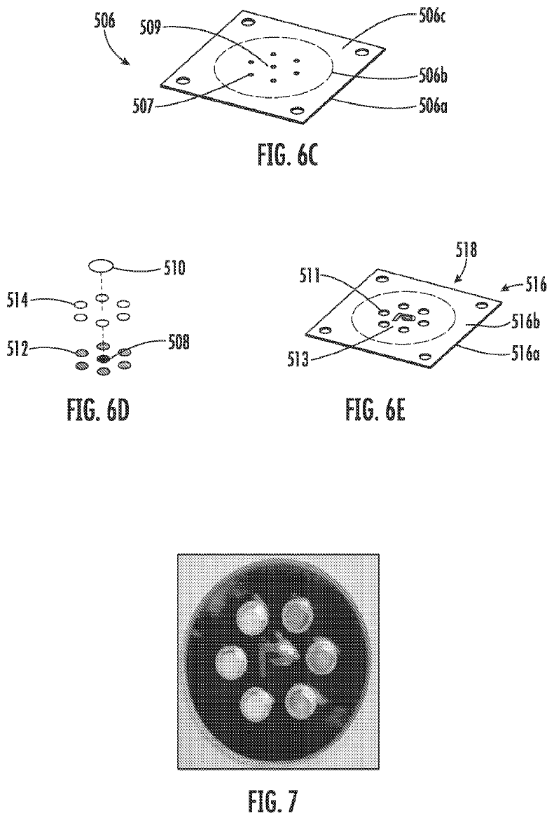

FIG. 6A shows a more detailed schematic of the substrate layer 502. It can be seen that substrate layer 502 in this example can comprise a plurality of inlets 501 that are in fluid communication with the device wearer's skin. It is further understood that the plurality of inlets 501 are in fluid communication with the device wearer's skin and in fluid communication with the at least one sweat collector 504. It is understood that the plurality of inlets can comprise any amount of inlets that can be needed for the desired application. For example, and without limitations, the device 500 comprises 18 inlets. It is understood that these inlets extend through the substrate layer 502. It is further understood that each of the inlets 501 can have any desired shape of orifice. The inlet 501 can be fully circular, substantially circular, square, rectangular, having an irregular form, etc. There is no specific limitation for the shape of the orifice of any one of the plurality of the inlets 501. In some aspects, the shape and size of each of the plurality of the inlets 501 are the same. However, in other aspects, the shape and size of each of the plurality of the inlets 501 can be different.

It is further understood, however, that the size of each of the plurality of inlets 501 can be adjusted to ensure that the total dead volume of the device 500 is reduced while still ensuring the simplicity of the manufacturing.

The number of the plurality of inlets 501 can also be adjusted to ensure that sweat is collected at a sufficient rate.

The substrate 502, adhesive layer 502a, and impermeable layer 502b of device 500 shown in FIG. 5, for example, have 18 round inlets 501 of approximately 1 mm diameter to allow sweat from the skin to access the upper layers of the device 500. Again, it is understood that the number and the size of each of the inlets can vary and the disclosed herein numbers are only exemplary and non-limiting.

In some exemplary aspects, as shown in device 500, the radial placement of the inlets 501 is towards the edge of the device 500, leaving sufficient space between the inlets and the edge of the device 500 to form a fluidic seal while leaving sufficient space between the inlets and a center of the device to position sensing elements (at least one sensor portion) 514 and a ready reagent 508. In such aspects, the plurality of inlets 501 present in the substrate 502 are placed to ensure fluidic contact with the subject's skin.

The placement of the inlet 501 on the substrate 502 can be done in any form as it can be needed for a specific application. The exemplary and unlimiting device 500 has a plurality of inlets 501 angularly disposed in six (6) clusters of three (3) to enable collection from a large enough total area of the skin.

In still further aspects, the device 500 comprises at least one sweat collector element 504 (also shown in FIG. 6B). This exemplary sweat collector element 504 comprises a central portion 515 and a plurality of channels 505 outwardly and radially extending from the central portion 515, wherein each of the plurality of channels 505 is in fluid communication with each other through the central portion 515.

In still further aspects, each of the plurality of channels has a distal portion 517 that is opposite from the central portion 515. In still further aspects, each of the channels 515 can be terminated with one or more inlets 503 configured to be in fluid communication with the substrate 502. The inlets 503 refer to a location where the sweat collector element 504 comes into intimate contact (e.g., fluid contact) with the plurality of inlets 501 of the substrate 502. In this specific example, the plurality of inlets 503 for the sweat collector 504 can be made of the same material as a plurality of channels 505 or any other material that can collect sweat aliquot coming from the substrate and transfer it through the channels 505 to the central portion 515 of the collector 504.

In certain aspects, each of the channels 505 can be terminated with two or more inlets 503 fluidically connected to each other and to the channels 505. The exemplary device 500, as shown in FIG. 6B, for example, has at least three inlets 503 fluidically connected to each other and to the channels 505. As discussed in detail above, these inlets 503 of the plurality of channels 505 are substantially aligned with the corresponding inlets 501 present in the substrate 502.

In still further aspects, the sweat aliquot collected by each inlet 503 of the plurality of channels 505 is transferred to the central portion 515 of the at least one sweat collector element.

It is understood that the sweat collector element 504 can have any shape and that the shape disclosed herein is only exemplary. In certain aspects, the shape of the sweat collector 504 can be a “snowflake” shape. Yet, in other aspects, the sweat collector element 504 can be any shape that can collect sweat from multiple locations and direct the sweat towards the central region.

It is understood that each of the plurality of inlets 501 present on the substrate 502 has a size that is the same or different and is effective to transfer the sweat aliquot to the one or more inlets 503 of the plurality of channels 504 by a capillary movement.

It is understood that a number of the plurality of channels 505 and a corresponding plurality of the inlets 503 on the sweat collector 504 can be defined by the number of the plurality of inlets 501 on the substrate 502 and the number of the sensing elements (sensor portions) 514 as disclosed below.

It is understood that the sweat collector of device 500 can comprise any materials disclosed above that can be used to make the sweat collector 504. Any materials that allow fluid movement via capillary forces can be used. In some aspects, paper can be used to form sweat collector 504. In yet other aspects, a filtering paper having different pore sizes can be utilized to form the sweat collector element 504. It is understood that the total volume of the sweat collector element 504 is substantially minimized such that the volume that must be filled with sweat before it can reach the sensor portion of the device is reduced. In still further aspects, the same reduction in volume can be controlled by, e.g., reducing the amount of material used to form the sweat collector element or by impregnating portions of the element with a non-porous, non-soluble material (such as wax).

In still further aspects, the device 500 can comprise a blocking layer 506, as shown in FIG. 5 and FIG. 6C. In still further aspects, the blocking layer 506 has a bottom portion facing the sweat collector element 504 and a top portion opposite to the bottom portion. The bottom portion of the layer 506 has an adhesive material 506a disposed thereon. While the top portion of the layer 506 has an impermeable polymer 506b disposed thereon. It is understood that this impermeable polymer defines the blocking layer 506 behavior.

In still further aspects, the blocking layer 506 is disposed on at least a portion of the at least one sweat collector element 504, wherein the blocking layer 506 comprises a plurality of apertures 507, wherein a first aperture of the plurality of apertures 509 is aligned with the central portion 515 of the at least one sweat collector element 504, and wherein remaining apertures 507 are radially and outwardly disposed from the first aperture 509 and aligned with at least a portion of each channel 505 of the at least one sweat collector element 504 to allow fluid communication with the at least one sweat collector element 504.

Again, and as disclosed above, the apertures 507 present in the blocking layer 506 can have any shape and size. In certain aspects, the apertures 507 have the same or different shape and size as the plurality of inlets 501 in the substrate layer 502. In certain aspects, the size of the apertures 507 can be chosen such that the volume required to be filled with sweat is reduced. Yet, in other aspects, the size of the apertures 507 can be chosen, such chances of fluid moving from upper to lower layers are also reduced. Still, in further aspects, the size is chosen for ease of manufacturing.

In certain aspects, the apertures 507 can have a circular form of about 1 mm in diameter. In certain aspects, the number of apertures 507 can be chosen depending on the number of sensing elements (sensor portions) 514. In still other aspects, the number of apertures 507 can be chosen depending on the number of the inlets 501 in the substrate 502 or the number of the inlets 503 in the sweat collector element 504. In the exemplary aspect shown in FIG. 5 and FIG. 6C, the blocking layer 506 can have six (6) circumferential apertures 507 and one aperture 509. In such aspects, the construction of this blocking layer 506 can allow a movement of the sweat aliquot from the sweat collector element 504 towards the sensing elements (sensor portions) 514 of the device 500. The specific positioning of the apertures 507 can vary. In some aspects, the positioning can be substantially evenly distributed in a concentric ring around the central aperture 509. In yet other aspects, and as disclosed above, the apertures 507 in the blocking layer 506 and the plurality of inlets 503 in the sweat collector element 505 are substantially aligned.

In still further aspects, the device 500 can further comprise a plurality of asymmetric membranes 512 as shown in FIG. 6D that are not in fluid communication with each other and are aligned with radial apertures 507 of the blocking layer 506 to allow fluid communication between asymmetric membranes 512 and the at least one sweat collector element 504. It is understood that the asymmetric membranes 512 can comprise any materials that allow a unidirectional flow. In these exemplary aspects, each of the plurality of asymmetric membranes 512 is porous and has an axial gradient in pore size to allow a unidirectional axial flow of the sweat aliquot. In certain aspects, the asymmetric membranes 512 can have a gradient in pore size from small to large, oriented so that small pores are on the upper surface. In such a configuration, the backflow of the sweat aliquot is limited.

It is understood that these asymmetric membranes 512 can be of any size and shape.

In still further aspects, and as shown in FIG. 5 and FIG. 6D, the device 500 comprises the plurality of sensing elements (sensor portions) 514. In such exemplary aspects, the plurality of sensing elements 514 are not in fluid communication with each other. As it can be seen in FIG. 6D, these sensing elements 514 are disposed above the plurality of asymmetric membranes 512. It can be further understood that the sensing elements 514 are in fluid communication with the plurality of asymmetric membranes 512.

In still further aspects, each of the plurality of sensing elements 514 can comprise a colorimetric reagent configured to react with at least one characteristic of the sweat. Any of the disclosed herein colorimetric reagents can be used in this exemplary device. The colorimetric reagent used in this device, similar to all devices disclosed above, can be present in an amount effective to determine a dynamic range of a concentration of the at least one characteristic of the sweat. While in other aspects, the colorimetric reagent used in this device can be present in an effective composition to determine a dynamic range of a concentration of the at least one characteristic of the sweat. In yet other aspects, the effective composition of the colorimetric reagent in each of the plurality of sensing elements can be different and is configured to determine a different concentration of the at least one characteristic of the sweat. It is understood that in the aspects where the colorimetric reagent comprises various components, a ratio between these different components of the colorimetric reagent can ensure the sensitivity of the reagent to different concentrations of the at least one characteristic of sweat. In still further aspects, the relative ratio of components in the colorimetric reagent in each paper element is selected to maximize the color change gradient around the target analyte concentration (i.e., change appearance completely, as visualized by a human eye, over the shortest analyte concentration range).

It is understood that each of the sensing elements 514 can have the same colorimetric reagent or different reagents. In yet other aspects, and as disclosed above, the colorimetric reagent can comprise the same components but have various ratios thereof. It is further understood that the specific choice of the reagents in these sensor segments 514 can be chosen such that the at least one characteristic of the sweat can be determined by visual inspection of the sensor segments 514. The choice of concentration and ratio of reagents in each of the sensor segments 514 can also be dependent on the volume of sweat absorbed by each of the sensor segments 514.

In some aspects, the sensor segments 514 can be positioned in the form of a clock dial such that there is a clockwise progression of sensing elements 514 that react to increasing concentrations of at least one characteristic of the sweat.

In still further aspects, the device 500 can further comprise an asymmetric indicator membrane 508 having a bottom portion and an upper portion. The bottom portion of the asymmetric indicator membrane 508 is positioned above the first aperture 509 of the blocking layer 506. The asymmetric indicator membrane 508 is also in fluid communication with the at least one sweat collector element 504 and is configured to transfer the sweat aliquot received from the first aperture 509 axially.

In such exemplary aspects, the asymmetric indicator membrane 508 can be any material having an axial gradient. For example and without limitations, the asymmetric indicator membrane 508 can be a porous membrane having an axial gradient in pore size, wherein the bottom portion of the asymmetric indicator membrane has a larger pore size than an upper portion, and wherein the upper portion of the asymmetric indicator membrane comprises a water-soluble dye configured to be dissolved in the sweat aliquot. It is understood that any water-soluble dies can be used. For example, and without limitations, Brilliant Blue FCF can be utilized. In still further aspects, an additional asymmetric porous membrane 510 is positioned above the asymmetric indicator membrane 508, collectively referred to as the “ready indicator.”

In still further aspects, the dye can be applied to the side of the asymmetric membrane 508 with the smaller pore size in order to constrain the position of the dye due to the preferential wicking of the membrane.

In still further aspects, the asymmetric indicator membrane 508 is positioned to receive the sweat aliquot after the colorimetric reagent present in each of the plurality of sensing elements 514 reacted with the at least one characteristic of the sweat. When device 500 is in use, sweat is collected from the skin by the sweat collector element 504. Sweat travels laterally in the sweat collector element 504 and then vertically into the central asymmetric membrane 508, where the dye dissolves into the sweat. The sweat then continues to move vertically into the porous material 510 above. By allowing the dye to dissolve in the sweat prior to it entering the porous material, it becomes visually apparent when sweat does enter the porous material. This positioning also ensures that a complete change in color of the porous material indicates that the whole patch has been saturated with sweat, and therefore the patch is ready to be read to determine the concentration of the target analyte.

In still further aspects, the asymmetric indicator membrane 508 is configured to indicate that the at least one characteristic of the sweat is determined.

The device 500 can further comprise a masking layer 516, as shown in FIG. 6E. The masking layer can comprise an adhesive layer 516a and an impermeable layer 516b. A top surface 518 of the masking layer can be patterned to form any shape or design such that a window to view a color change of the sensor segments 511 and a window to view ready indicator 513 are formed. In some aspects, the masking layer can comprise a symbol, logo, or text that is configured to be visually observed when the at least one characteristic of the sweat is determined. Any of the disclosed above phrases can be shown on the masking layer of this device.

It is understood that in some aspects, the geometry of the device can be changed. It is understood, however, that the change should not affect that a volume of material between the sweat collection points and the detection dots is equal. In still further aspects, the variation in device geometry should ensure that there is no flow of sweat from between the sensor segments or between the sensor segments and the ready indicator. In still further aspects, the geometry of the device ensures that the volume of material between the sensor segments and the ready indicator is enough to allow the sensor segments to fully saturate before the ready indicator is triggered.

In general, the use of asymmetric membranes can be optional. However, it is understood that the unidirectionality of these membranes helps to prevent cross-contamination.

FIG. 7 shows an exemplary visual reading obtained by device 500 in the experimental assembly shown in FIG. 3. FIG. 9 shows the change of color with increasing analyte concentration in sweat. The solid line represents the preferred change.

In still further aspects, the device is configured to detect natural sweating, medically induced sweating, or a combination thereof. In certain aspects, the device disclosed herein can indicate the subject's level of hydration or whether the subject has any medical conditions affecting their sweat.

In certain aspects, the devices disclosed herein can be in communication with smart devices. For example, the device can also comprise an RFID tag that can be read by a smart device. Yet, in other aspects, the measurements can be transferred to the smart device with the use of a camera and cataloged for a short and/or long-term observation of the sweat characteristics.

In yet further aspects, the device described herein is not limited to measurement of the sweat and can be adapted for measurement of any other appropriate fluid, whether it is a biological fluid or not. In such aspects, the biofluid, for example, can comprise sweat, saliva, blood, urine, tears, reproductive fluids, or any combination thereof. While in other aspects, the non-biofluid can comprise tap water, lake water, ocean water, wastewater, rain, water, groundwater, industrial process fluids, water from swimming pools, beverages, raw ingredients, or any combination thereof.

In still further aspects, the devices disclosed herein are disposable.

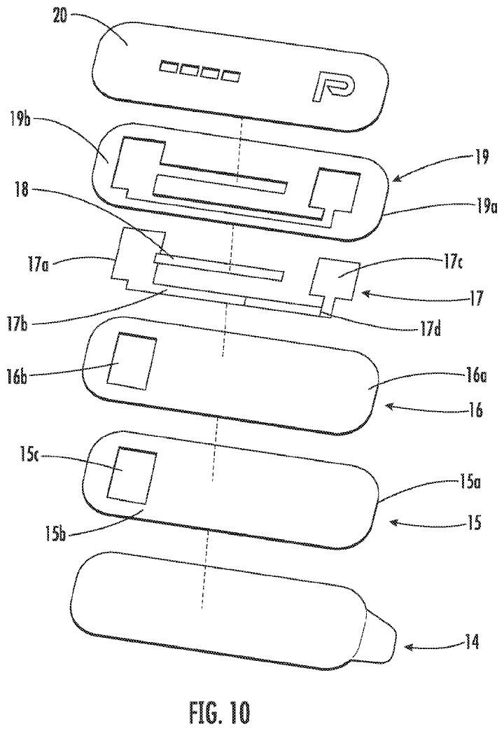

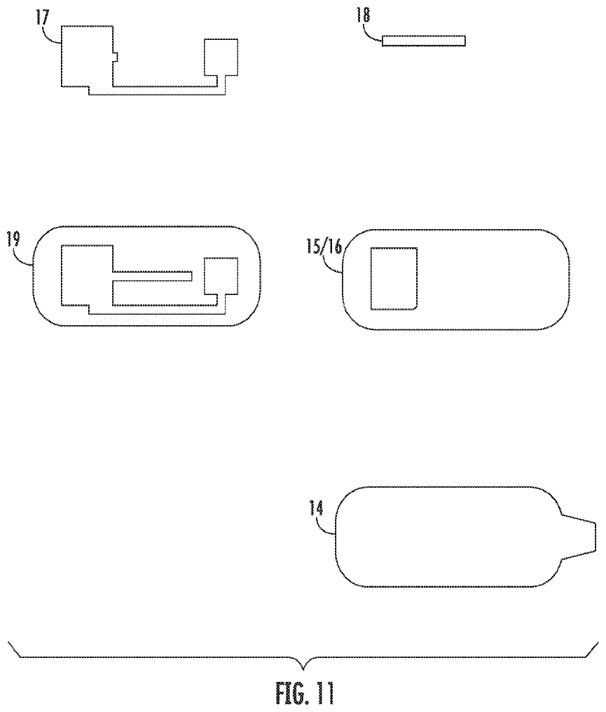

An additional exemplary aspect of the present disclosure is disclosed in FIGS. 10 and 11. In these exemplary and unlimiting aspects, the device can have a rectangular form. Again, it is understood, however, that such a rectangular form is only exemplary, and any other shapes can be used if needed or convenient. In some exemplary aspects, the shape can be square, rhombic, triangular, irregular shape, trapezoid shape, or any other shape.

Similar to an exemplary device shown in FIG. 1, the device shown in FIG. 10 can have any desired dimensions. It is understood, however, that in general, the device can be sized to minimize the volume of sweat that is required to be collected and to enable a reduction of the time to saturation and thus to minimize the time for receiving the measurement result. But it is also understood that the smallest dimension of the device will be limited by the ability of the wearer to determine the measurement results. Yet, in other aspects, it is also understood that having large devices can be inconvenient to the wearer and not economically beneficial. Also, it is understood that the larger device can require a longer time for the sweat collection and, as a result, a longer time for receiving the results that can also be imprecise due to undesirable sweat evaporation.

In still further aspects, the size of the exemplary device as shown in FIG. 10 can be anywhere between about 25 mm to about 1 cm in length, including exemplary values of about 30 mm about 35 mm, about 40 mm, about 45 mm, about 50 mm, about 55 mm, about 60 mm, about 65 mm, about 70 mm about 75 mm, about 80 mm, about 85 mm, about 90 mm, and about 95 mm.

Yet in other aspects, the size of the exemplary device can be anywhere between about 1 mm to about 50 mm in width, including exemplary values of about 1.5 mm, about 2 mm, about 2.5 mm, about 3 mm, about 3.5 mm, about 4 mm, about 4.5 mm, about 5.5 mm, about 6 mm, about 6.5 mm in width, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm in width, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 22.5 mm, about 23 mm, about 23.5 mm, about 24 mm, about 24.5 mm, about 25 mm, about 25.5 mm, about 26 mm, about 26.5 mm in width, about 27 mm, about 27.5 mm, about 28 mm, about 28.5 mm, about 29 mm, about 29.5 mm, about 30 mm, about 30.5 mm, about 31 mm, about 31.5 mm, about 32 mm, about 32.5 mm, about 33 mm, about 33.5 mm, about 34 mm, about 34.5 mm, about 35 mm, about 35.5 mm, about 36 mm, about 36.5 mm in width, about 37 mm, about 37.5 mm, about 38 mm, about 38.5 mm, about 39 mm, about 39.5 mm, about 40 mm, about 40.5 mm, about 41 mm, about 41.5 mm, about 42 mm, about 42.5 mm, about 43 mm, about 43.5 mm, about 44 mm, about 44.5 mm, about 35 mm, about 35.5 mm, about 36 mm, about 36.5 mm in width, about 37 mm, about 37.5 mm, about 38 mm, about 38.5 mm, about 39 mm, and about 39.5 mm.

In still further aspects, an overall thickness of the device can be anywhere between 50 μm to about 500 μm, including exemplary values of about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 305 μm, about 310 μm, about 315 μm, about 320 μm, about 325 μm, about 330 μm, about 335 μm, about 340 μm, about 345 μm, about 350 μm, about 355 μm, about 360 μm, about 365 μm, about 370 μm, about 375 μm, about 380 μm, about 385 μm, about 390 μm, about 395 μm about 400 μm, about 405 μm, about 410 μm, about 415 μm, about 420 μm, about 425 μm, about 430 μm, about 435 μm, about 440 μm, about 445 μm, about 450 μm, about 455 μm, about 460 μm, about 465 μm, about 470 μm, about 475 μm, about 480 μm, about 485 μm, about 490 μm, and about 495 μm.

The devices described herein can comprise multiple stacked layers to add additional functionalities or protect components and reduce the risk of extraneous environmental fluids entering the system. The devices may have a tapered geometry that increases the adhesion to the skin, promotes the formation of a seal to prevent extraneous liquid from reaching the inlet, and reduces the risk of delamination. The described devices can be flexible, stretchable, and can establish conformal contact with the underlying skin surface.

As shown in FIG. 10, the device can comprise a substrate configured to be removably attached to a subject's skin, wherein the substrate has at least one inlet in fluid communication with at least the subject's skin and wherein the at least one inlet is configured to receive and transfer a sweat aliquot. The substrate 15 is in contact with the subject skin, as shown in FIG. 10, has at least one inlet 15c that can collect, receive and transfer a sweat aliquot to at least one sweat collector element 17a. The substrate can have an adhesive layer 15a disposed on a skin-facing surface of the substrate. Before the use of the device, the adhesive layer 15a can be covered with a protective layer 14 that can be easily peeled off to allow a quick attachment to the subject's skin.

It is understood that the at least one inlet 15c can have any shape and any area that are suitable for the collection of the desired amount of sweat in the desired amount of time. In certain aspects, the at least one inlet 15c can have a circular shape, a rectangular shape, a square shape. It can also have a rhombic, triangular, irregular shape, trapezoid shape, or any other shape that can provide a flow path such that sweat can reach at least one sweat collector element 17a in the desired amount of time. In some exemplary and unlimiting aspects, the at least one inlet can have an area from about 50 mm² to about 200 mm², including exemplary values of about 60 mm², about 70 mm², about 80 mm², about 90 mm², about 100 mm², about 110 mm², about 120 mm², about 130 mm², about 140 mm², about 150 mm², about 160 mm², about 170 mm², about 180 mm², and about 190 mm².

In still further aspects, the substrate 15 can be made from any materials. For example, it can comprise impermeable polymer 15b. However, any known in the art materials that are not permeable to the sweat can be used. For example, and without limitations, the substrate can comprise silicone, polyurethane, polyvinyl chloride (PVC), polyethylene terephthalate (PET), thermoplastic elastomers, nylons, metalized-PET, waxed papers or modified waxed papers, polydimethylsiloxane (PDMS), polyurethane sponge, polyvinyl alcohol sponge, silicone sponge, polystyrene, polyimide, SU-8, wax, olefin copolymer, polymethyl methacrylate (PM MA), polycarbonate, poly(styrene-isoprene-styrene), chitosan, and any combination thereof. In certain aspects, the substrate comprises any materials that are non-absorbing and have a substantially moderate-to-low water vapor transfer rate.

It is understood that peel-off layer 14 can be made of any known in the art materials that can protect and easily be removed. For example, and without limitations, the peel-off material can comprise bleached kraft paper, unbleached kraft paper, waxed papers, modified waxed papers, or polyethylene terephthalate films.

In certain aspects, an additional layer 16 can be disposed on the substrate 15. In such aspects, the layer 16 can be an opaque impermeable layer. In certain aspects, an adhesive layer 16a is used to attach layer 16 to the substrate. The layer 16 can also have at least one opening 16b that is substantially identical to at least one inlet 15c that allows the transfer of the sweat aliquot from the subject's skin to the at least one sweat collector 17a. It is understood, however, that the at least one opening 16b does not have to be substantially identical to the at least one inlet 15c. In certain aspects, the opening 16b can have a shape that is the same or different from the at least one inlet 15c. In yet other aspects, the opening 16b can have an area that is the same or different from the area of the at least one inlet 15c. In certain aspects, the area of the opening 16b can be bigger than the area of the at least one inlet 15c. While in other aspects, the opening 16b can have an area that is smaller than the area of the at least one inlet 15c.

In still further aspects, the opaque impermeable layer 16 can comprise any known in the art materials suitable for the desired application. For example, it can be an opaque white vinyl(polyvinyl chloride) sheet. However, it is understood that any material that can provide enough opacity to hide the internal components from view and five a uniform color background could be used. It is understood that layer 16 can prevent the sweat aliquot from evaporating or, in any other way, affect the measurement reading. Any of the impermeable polymers or materials disclosed herein can be used for this purpose.

In still further aspects, the device can comprise sensoring layer 17. In such aspects, the sensoring layer can comprise the at least one sweat collector 17a. In these exemplary aspects, the sweat collector is shown as having a rectangular form. However, it is understood that the at least one sweat collector element can have any shape and any dimensions suitable to collect and transfer the desired amount of sweat. For example, and without limitations, the at least one sweat collector element can have a circular shape, square, rhombic, triangular, irregular shape, trapezoid shape, or any other shape. In yet still further exemplary aspects, the at least one sweat collector can have an area from about 50 mm² to about 200 mm², including exemplary values of about 60 mm², about 70 mm², about 80 mm², about 90 mm², about 100 mm², about 110 mm², about 120 mm², about 130 mm², about 140 mm², about 150 mm², about 160 mm², about 170 mm², about 180 mm², and about 190 mm². The sweat collector 17a is configured to receive the sweat aliquot from at least one inlet 15c on a substrate.

In some aspects, the at least one sweat collector element 17a comprises a porous material configured to directionally adsorb and transfer the sweat aliquot. In yet further aspects, the porous material can comprise one or more layers. In still further aspects, the at least one sweat collector 17a element can comprise a paper, a polymer, a textile, a foam, or any configuration thereof.

In still further aspects, the device can further comprise at least one sensor element 18 having a proximal end and a distal end, a first length, l₁, along a longitudinal axis of the device, measured between the proximal end and the distal end of the at least one sensor element, wherein the at least one sensor element is in fluid communication with the at least one sweat collector element; and wherein the at least one sensor element is configured to quantitatively detect at least one characteristic of the sweat.

In still further aspects, the at least one sensor element that is in fluid communication with the at least one sweat collector element can have any first length, l₁, that is compatible with the device size and application. Again, it is understood that the terms “proximal” and “distal” as used herein are relative terms to describe each of the sensor element terminals relative to each other. In this exemplary aspect, the proximal end of the at least one sensor element is in closer proximity to the at least one sweat collector element, and the distal end is disposed further away from the at least one sweat collector element along the longitudinal axis of the device. In some aspects, the first length can be anywhere between about 10 mm to about 30 mm, including exemplary values of about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, and about 29 mm.

In still further aspects, the positioning of the at least one sensor element can be anywhere relative to the at least one sweat collector element as long as there an efficient fluid communication between two of these elements.

In still further aspects, the device further comprises at least one ready indicator element 17c that is in fluid communication with the at least one sweat collector element 17a through at least one fluidic path 17b having a proximal end and a distal end and having a second length, l₂, as measured between the proximal and distal ends along the longitudinal axis of the device, wherein the second length is greater than the first length (l₂>l₁). Again, it is understood that the terms proximal and distal ends in relation to the fluidic path 17b are relative and are used to indicate the position of each terminal relative to each other.

In certain aspects, at least a portion of the at least one sweat collector element extends into the proximal end of the at least one fluidic path. In still further aspects, the distal end of the at least one fluidic path 17b can extend into a portion of the at least one ready indicator element 17c. It is understood that the at least one ready indicator element that is also in fluid communication with the at least one sweat collector element can be positioned anywhere relative to the at least one sweat collector element and the at least one sensor element.

In some aspects, the second length, l₂, can be any length that allows achieving the desired results. In some aspects, the second length l₂ can be anywhere between about 10 mm to about 50 mm, including exemplary values of about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, and about 49 mm.

In still further aspects, the at least one ready indicator element 17c can have a predetermined shape having a predetermined area and is positioned substantially parallel to the at least one sweat collector element. Again, it is understood that also disclosed are aspects where the at least one ready indicator element is not positioned substantially parallel to the at least one sweat collector element. It is understood that it can be positioned anywhere on the device and in any relation to the at least one sweat collector element or any other elements present in the sensoring layer 17. The at least one ready indicator element can have any of the disclosed above shapes.

In certain aspects, the predetermined shape and/or predetermined area of the at least one ready indicator element is the same or different from the predetermined shape and/or predetermined area of the at least one sweat collector element, respectively. Yet in other aspects, the predetermined area of the at least one ready indicator element can be anywhere between about 25 mm² to about 100 mm², including exemplary values of about 30 mm², about 35 mm², about 40 mm², about 45 mm², about 50 mm², about 55 mm², about 60 mm², about 65 mm², about 70 mm², about 75 mm², about 80 mm², about 85 mm², about 90 mm², and about 95 mm².

In still further aspects, the at least one fluidic path 17b is configured to transfer a first portion of the collected sweat aliquot from the at least one sweat collector element 17a to at least one ready indicator element 17c. In still further exemplary and unlimiting aspects, at least a portion of the at least one fluidic path can comprise a dye configured to dissolve in the first portion of the sweat aliquot and be transferred to the ready indicator element. It is understood that any dye known in the art and suitable for the desired application can be used. In some exemplary and unlimiting aspects, a dye such as Brilliant Blue FCF can be used. The dies can be inserted anywhere within the fluidic path. In some aspects, the dye is inserted abut the distal end of the fluidic path in proximity to the at least one ready indicator element. In such aspects, when a portion of the sweat aliquot riches the dye, the dye dissolves in the sweat. The dissolved dye is then carried to the at least one ready indicator that shows to the user that the device is ready to read the measurement.

In still further aspects, the at least one ready indicator element 17c comprises a porous material configured to directionally adsorb the sweat aliquot and the dye. In yet further aspects, the porous material can comprise one or more layers. In still further aspects, the at least one ready indicator element 17c element can comprise a paper, a polymer, a textile, a foam, or any configuration thereof.

It is understood that both the at least one sensor element and at least one fluidic path can comprise a porous material configured to directionally adsorb the sweat aliquot. In yet further aspects, the porous material can comprise one or more layers. In still further aspects, the at least one sensor element and at least one fluidic path can comprise a paper, a polymer, a textile, a foam, or any configuration thereof.

In still further aspects, the sweat collector element, at least one sensor element, at least one ready indicator element can occupy all the width and/or length of the device, or it can occupy a portion of the device. In some aspects, the sweat collector element, at least one sensor element, at least one ready indicator element can be centered on the substrate of the device. While in other aspects, the sweat collector element, at least one sensor element, at least one ready indicator element they can be positioned anywhere on the device. Yet, in other aspects, the arrangement of the component can be determined by the specific application or manufacturing methods.

In certain aspects, the at least one sensor element 18 is coupled to the at least one sweat collector element, such that it is positioned above the at least one fluidic path. It is understood, however, that the term “coupled” as used herein can be applied to the devices where the at least one sweat collector element and the at least one sensor element are manufactured separately and then physically connected and to the devices where the at least one sweat collector element and the at least one sensor element are manufactured as a single piece extending to each other.

In still further aspects, the at least one sensor element can be coupled to the at least one sweat collector element, such that it is positioned parallel to the at least one fluidic path. However, this parallel position is not mandatory, and the at least one sensor element can be positioned anywhere relative to the at least one fluidic path.

In still further aspects, the at least one sensor element 18 comprises a reagent configured to react with the at least one characteristic of the sweat.

In some aspects, the reagent is a colorimetric reagent configured to change an original color of the reagent upon reaction with the at least one characteristic of the sweat. However, it is also understood that the reagent can be any reagent that can react with the at least one characteristic of the sweat to produce a product or a different reagent that can be correlated with the presence of the at least one characteristic. For example and without limitation, the reaction between the reagent and the at least one characteristic of the sweat can result in a fluorescent, photo fluorescent, or luminescent signal. It can also result in a response that can be measured in the UV or IR spectrum of the light. Yet, in other aspects, when the colorimetric reagent is used, it shows a change in color in a visible spectrum providing the device's wearer with quick identification of the measurement.

In still further aspects, any known in the art colorimetric reagents can be sued. For example, and without limitation, the colorimetric reagent can comprise silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion or iron ion, a 2,2-bicinchoninic acid, a, 10-phenanthroline, a universal pH indicator, silver dichromate, a compound of fluorescein and silver nitrate, or any combination thereof. In still further aspects, the colorimetric reagent is 2,4,6-tris(2-pyridiyl)-s-triazine (TPTZ) complexed with mercury ion, silver ion, or iron ion; or silver chloroanilate. In still further exemplary and unlimiting examples, the at least one sensor element of the devices disclosed herein, such as, for example, 18, can comprise 4,6-tris(2-pyridiyl)-s-triazine (TPTZ) complexed with mercury ion, silver ion, or iron ion; or silver chloroanilate.

In still further aspects, the colorimetric reagent can be present in an amount effective to determine a dynamic range of a concentration of the at least one characteristic of the sweat. While in other aspects, the colorimetric reagent can be present in an effective composition to determine a dynamic range of a concentration of the at least one characteristic of the sweat. In such exemplary aspects, the reagent can comprise various compositions and include more than one component, and therefore the dynamic range of the concentration of the at least one characteristic of the sweat can also be dependent on a ratio of these components within the reagent.

It is understood that the reagent can be impregnated within the at least one sensor element from a solution, a suspension, or formed in-situ by reaction with the at least one characteristic of the sweat or by a printing method.

It is understood that the concentration of the reagent can be the same or different throughout the element 18. In some aspects, the amount of the colorimetric reagent can be substantially similar per unit area. While in other aspects, the sensor element 18 can have a plurality of portions having different concentrations of the reagent.

It is understood that the specific chemistry of the reagent in the at least one sensor element 18 can be selected such that the length of the element that changes color can show the most suitable dynamic range for the purpose (i.e., no color change occurs at very low analyte concentrations while the entire length of the element changes color at the highest expected analyte concentrations). In such exemplary aspects, the at least one characteristic of the sweat is exposed to an increasing cumulative amount of reagent as the sweat aliquot proceeds from the proximal end of the at least one sensor element to its distal end. In yet other aspects, the sensor element 18 is configured to indicate a concentration of the at least one characteristic of the sweat by a substantial change of color of the colorimetric reagent from its proximal end to its distal end. In some aspects, the change of color is substantially linear. While in other aspects, the change of color is linear. While in still further aspects, the change of color is substantially non-linear. In yet further aspects, the change of color is at least partially linear or partially non-linear. In still further aspects, the change of color can achieve saturation.

It is understood that the mechanism of the reaction between the reagent and the at least one characteristic of the sweat is dependent on the reagent and the specific characteristic. It is further understood that the reagent can be disposed along a length of the sensor element 18. As discussed in detail herein, the reagent can have the same concentration per unit area of the sensor element or different depending on the desired application. In certain aspects, the sweat aliquot can travel along the at least one sensor element such that the at least one characteristic of the sweat reacts with the reagent present in the sensor element 18 at its first point of contact, removing at least a portion of the at least one characteristic of the sweat from the sweat. As more sweat enters the sensor element, it needs to travel further along the sensor element before it encounters an un-reacted reagent left in the sensor element that can react with at least one characteristic of the sweat in the new aliquot causing a change in color. Again, as discussed above, the change can be substantially linear, partially linear, substantially non-linear, or partially non-linear.

In still further aspects, the at least one sensor element can have a first width, d₁. It is understood that the first width can be any width that allows achieving the desired results. In some aspects, the first width can be anywhere between about 1 mm to about 5 mm, including exemplary values of about 1.5 mm, about 2 mm, about 2.5 mm, about 3 mm, about 3.5 mm, about 4 mm, and about 4.5 mm. In still further aspects, the first width, d₁, can be the same or different along the first length, l₁.

In some aspects, the at least one fluidic path has a second width, d₂. In such exemplary aspects, the second width, d₂, is the same or different along the first length, l₂. In yet further aspects, the second width d₂ is the same as the first width d₁ (d₁=d₂) or wherein the second width d₂ is smaller than the first width d₁ (d₁>d₂). Also disclosed are aspects where the second width d₂ is greater than the first width d₁ (d₁<d₂). In yet further aspects, the second width d₂ can be anywhere between about 1 mm to about 5 mm, including exemplary values of about 1.5 mm, about 2 mm, about 2.5 mm, about 3 mm, about 3.5 mm, about 4 mm, and about 4.5 mm.

In still further aspects, the devices disclosed herein show substantially no backflow between the at least one sensor element and the at least one fluidic path.

In still further aspects, the device can comprise more than one sweat collector element. In yet other aspects, the device can comprise more than one inlet. In still further aspects, the device can comprise more than one sensor element. In yet still further aspects, the device can comprise more than one fluidic path. In yet other aspects, the device can comprise more than one ready indicator element.

For example, and without limitations, if multiple sensor elements are present, such sensor elements can also have different concentrations of the reagent. In such aspects, the accuracy and precision of readings can also be improved. However, it is also understood that consideration needs to be given to a potential increase in the volume of sweat required to be absorbed by the device.

In still further aspects, the device can also comprise an occlusive impermeable layer 19. Such a layer can prevent the dye, the reagent, the sweat aliquot, or a reaction product between the reagent and at least one characteristic of the sweat aliquot from evaporating or in any other way to affect the measurement reading. The impermeable layer 19 has an adhesive layer 19a disposed on a bottom portion of the impermeable layer that assists in adhering the impermeable layer 19 to the underlying layers of the device and an impermeable polymer layer 19b disposed on a top portion. Any of the impermeable polymers or materials disclosed herein can be used for this purpose.

As shown in FIG. 10, the device can further comprise a patterned upper surface or indicator layer 20. It is understood that this layer is optional. In some aspects, this layer can be helpful to the device wearer to read and understand the measurement results more clearly. In such aspects, the indicator layer can also show when the device is ready for reading. In some aspects, the indicator layer 20 can be disposed on the at least one sensor element 18 and configured to visually segment the presence of the at least one characteristic of the sweat. The indicator layer can have windows, for example, to view the color change. Additional indicator layers are disclosed above with respect to the device of FIG. 1 and FIG. 2. It is understood that the indicator layer can be used to indicate which color the element should start and/or finish to assist users in determining if a portion of the element has or hasn't changed color. The indicator layer can have any shape, structure, or design. For example, the windows that correlate to a specific amount of the measured characteristic can be represented by a logo or by a specific phrase, or dots, or stars, or any other shapes. For example, and without limitation, the segments can spell a word “DRINK,” a word “READY,” “EXPIRED,” “WARNING,” “ALERT,” or any other phrases or words that would bring the device wearer's attention to take or not to take a specific action.

In further aspects, the device can comprise at least one substantially transparent layer. In such aspects, the layer can comprise a material having UV and/or Blue light blocking characteristics. In such exemplary and unlimiting aspects, the material can minimize the effects of the exposure of the reagents to sunlight on the measurement. In still further aspects, this transparent layer can be a top layer of the device. In yet other aspects, the layer can be disposed in the sequences of layers as long as it does not affect the desired application and is capable of blocking UV and Blue light if desired.

The devices disclosed herein can measure any characteristics of the sweat. In some aspects, the at least one characteristic of the sweat can comprise an amount of cations, anions, pH, temperature, volume, an amount of biomarkers, or a combination thereof. Even further, the at least one characteristic of the sweat can be, for example, the amount of cations selected from sodium, potassium, calcium, magnesium, aluminum, iron, or a combination thereof. In yet still further aspects, the at least one characteristic of the sweat is the amount of anions selected from chlorides, lactates, or a combination thereof. In still further aspects, the biomarkers can comprise glucose, cholesterol, uric acid, urea, ascorbic acid, lactate, electrolytes, proinflammatory cytokines, hormones, tuberculosis-specific proteins, Parkinson-specific proteins, schizophrenia-specific proteins, cancer-specific metabolic markers, cystic fibrosis specific markers, drug-specific markers, alcohol, or any combination thereof. Exemplary hormone that can be measured by the disclosed devices comprises but is not limited to cortisol.

In some aspects, the at least one characteristic of the sweat can be present in an amount from about 0.010 mM to about 500 mM, including exemplary values of about 0.05 mM, about 0.07 mM, about 0.1 mM, about 0.2 mM, about 0.5 mM, about 1 mM, about 5 mM, about 10 mM, about 20 mM, about 30 mM, about 40 mM, about 50 mM, about 60 mM, about 70 mM, about 80 mM, about 90 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, and about 475 mM.

In still further aspects, the device can also comprise additional sensor elements configured to sense a biological response different from one characteristic of the sweat. In some aspects, the biological response comprises a heart rate, blood pressure, skin temperature, sweat rate, blood oxygen saturation, or any combination thereof.

In still further aspects, the device is disposable. In still further aspects, and as disclosed above, the device can also be configured to be in communication with a processing unit.

Also disclosed is a device comprising: a) a substrate having at least one inlet that is configured to receive and transfer a fluid aliquot; b) at least one collector element configured to receive the fluid aliquot from the at least one inlet of the substrate; c) at least one sensor element having a proximal end and a distal end, a first length, l₁, along a longitudinal axis of the device, measured between the proximal end and the distal end of the at least one sensor element, wherein the sensor element is in fluid communication with the at least one collector; and wherein the at least one sensor element is configured to quantitatively detect at least one characteristic of the fluid; d) at least one ready indicator element that is in fluid communication with the at least one collector through at least one fluidic path having a proximal end and a distal end and having a second length, l₂, as measured between the proximal and distal ends along the longitudinal axis of the device, wherein the second length is greater than the first length (l₂>l₁); and wherein the device is configured to indicate a presence of the at least one characteristic of the fluid, wherein the fluid is a biofluid or a non-biofluid. In such aspects, the biofluid comprises sweat, saliva, blood, urine, tears, reproductive fluids, or any combination thereof. In still further aspects, the non-biofluid comprises tap water, lake water, ocean water, wastewater, rain, water, groundwater, industrial process fluids, water from swimming pools, beverages, raw ingredients, or any combination thereof.

### Methods

Also disclosed herein are methods of making the disclosed devices. In certain aspects, the methods comprise a) providing a substrate configured to receive and transfer a fluid aliquot; b) disposing at least one fluid collector element such that is it in fluid communication with the substrate and has at least one inlet configured to receive the fluid aliquot; wherein the at least one fluid collector element has a fluidic configuration such that a flow of the fluid aliquot is directed along a longitudinal axis and/or an axial axis of the at least one fluid collector element; c) disposing at least one sensor portion that is in fluid communication with the at least one fluid collector element; wherein the at least one sensor portion is configured to quantitively detect at least one characteristic of the fluid; and wherein the device is configured to indicate a presence of the at least one characteristic of the fluid.

In still further aspects, each of the materials disclosed above can be used to form the device. The colorimetric reagent can be impregnated within the at least one sensor portion of the device from a reagent solution or from a reagent suspension. The colorimetric reagent can be printed on the at least one sensor portion of the device. In still further aspects, the colorimetric reagent can be formed in-situ.

It is understood, and as disclosed above, the devices disclosed herein can be constructed as one piece or as separate elements that are then assembled together. In certain aspects, the device can be printed. It is understood that the printing methods can include 2D printing. In yet other aspects, the elements can be formed by die cutting or laser cutting.

During the manufacturing process, in certain aspects, the chemistry can be added before the final sensor element or fluidic path is formed, or it can be added after the sensor element and the fluidic path are formed.

Also disclosed are methods of using the disclosed herein devices. In aspects wherein the characteristics of the sweat are measured, the device is attached to the skin such that it stays attached during the entire duration of the measurement. The sweat is then collected within the sweat collector element and then transferred toward the sensing portion. After the reaction of the sweat with the colorimetric reagent, the results are shown in the indicator layer of the device.

### Aspects:

Aspect 1: A device comprising: a) a substrate configured to be removably attached to a subject's skin, wherein the substrate is in fluid communication with at least the subject's skin and configured to receive and transfer a sweat aliquot; b) at least one sweat collector element disposed on the substrate such that it is in fluid communication with the substrate and has at least one inlet configured to receive the sweat aliquot; wherein the at least one sweat collector element has a fluidic configuration such that a flow of the sweat aliquot is directed along a longitudinal axis and/or an axial axis of the at least one sweat collector element; and c) at least one sensor portion that is in fluid communication with the at least one sweat collector element; wherein the at least one sensor portion is configured to quantitively detect at least one characteristic of the sweat; and wherein the device is configured to indicate a presence of the at least one characteristic of the sweat.

Aspect 2: The device of Aspect 1, wherein the substrate has a proximal portion and a distal portion, and wherein the substrate comprises an inlet disposed in the proximal portion of the substrate that is configured to receive and transfer the sweat aliquot to the at least one inlet of the at least one sweat collector element.

Aspect 3: The device of Aspect 1 or 2, wherein the at least one sweat collector element has a proximal portion and an opposite distal portion, wherein the at least one inlet is disposed at the proximal portion of the at least one sweat collector element.

Aspect 4: The device of Aspect 2 or 3, wherein the inlet of the substrate and the at least one inlet of the sweat collector element are substantially aligned.

Aspect 5: The device of Aspect 3 or 4, wherein the flow of the sweat aliquot is gradual and longitudinal from the proximal portion to the distal portion of the sweat collector element.

Aspect 6: The device of any one of Aspects 1-5, wherein the at least one sensor portion is in intimate contact with the sweat collector element.

Aspect 7: The device of Aspect 6, wherein the at least one sensor portion is at least partially embedded within the sweat collector element.

Aspect 8: The device of Aspect 6, wherein the at least one sensor portion is disposed in a separate layer that is in intimate contact with the sweat collector element.

Aspect 9: The device of Aspect 6, wherein one sensor portion is at least partially embedded within the sweat collector element and wherein a second sensor portion is disposed in a separate layer that is in intimate contact with the sweat collector element.

Aspect 10: The device of Aspect 7 or 9, wherein the sensor portion is embedded along a length of the sweat collector element between the proximal portion and the distal portion of the sweat collector element.

Aspect 11: The device of Aspect 8 or 9, wherein the separate layer with the sensor portion is disposed along a length of the sweat collector element between the proximal portion and the distal portion of the sweat collector element.

Aspect 12: The device of any one of Aspects 1-11, wherein the sensor portion comprises a colorimetric reagent configured to react with the at least one characteristic of the sweat.

Aspect 13: The device of Aspect 12, wherein the colorimetric reagent is present in an amount effective to determine a dynamic range of a concentration of the at least one characteristic of the sweat.

Aspect 14: The device of Aspect 12, wherein the colorimetric reagent is present in an effective composition to determine a dynamic range of a concentration of the at least one characteristic of the sweat.

Aspect 15: The device of Aspect 13 or 14, wherein the amount of the colorimetric reagent is substantially similar per unit area.

Aspect 16: The device of any one of Aspects 13-15 wherein the at least one characteristic of the sweat is exposed to an increasing cumulative amount of reagent as the sweat aliquot proceeds from the proximal portion of the sweat collector element to the distal portion of the sweat collector element.

Aspect 17: The device of any one of Aspects 13-16, wherein the sensor portion is configured to indicate a concentration of the at least one characteristic of the sweat by a substantial change of color of the colorimetric reagent from the proximal portion to the distal portion of the sweat collector element.

Aspect 18: The device of any one of Aspects 1-17, wherein the sweat collector element has a rectangular shape.

Aspect 19: The device of any one of Aspects 1-18, wherein the sweat collector element comprises a porous material configured to directionally adsorb and transfer the sweat aliquot.

Aspect 20: The device of Aspect 19, wherein the porous material comprises one or more layers.

Aspect 21: The device of any one of Aspects 1-20, wherein the sweat collector element comprises a paper, a polymer, a textile, a foam, or any configuration thereof.

Aspect 22: The device of Aspect 21, further comprising an indicator layer disposed on the sensor portion and configured to visually segment the presence of the at least one characteristic of the sweat.

Aspect 23: The device of any one of Aspects 1-22, wherein the at least one characteristic of the sweat comprises an amount of cations, anions, pH, temperature, volume, an amount of biomarkers, or a combination thereof.

Aspect 24: The device of Aspect 23, wherein the at least one characteristic of the sweat is the amount of cations selected from sodium, potassium, calcium, magnesium, aluminum, iron, or a combination thereof.

Aspect 25: The device of Aspect 23, wherein the at least one characteristic of the sweat is the amount of anions selected from chlorides, lactates, or a combination thereof.

Aspect 26: The device of any one of Aspects 1-25, wherein the at least one characteristic of the sweat is present in an amount from about 0.10 mM to about 500 mM.

Aspect 27: The device of any one of Aspects 12-26, wherein the colorimetric reagent comprises silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion or iron ion, a 2,2-ticinchoninic acid, a, 10-phenanthroline, a universal pH indicator, silver dichromate, a compound of fluorescein and silver nitrate, or any combination thereof.

Aspect 28: The device of any one of Aspects 12-27, wherein the colorimetric reagent is impregnated within the at least one sensor portion from a solution, a suspension, or formed in-situ by reaction with the at least one characteristic of the sweat, or by a printing method.

Aspect 29: The device of any one of Aspects 1-28, wherein the device is configured to provide a visual indication of the presence of the at least one characteristic of the sweat.

Aspect 30: The device of Aspect 1, wherein the at least one sweat collector element comprises a central portion and a plurality of channels outwardly and radially extending from the central portion, wherein each of the plurality of channels is in fluid communication with each other through the central portion.

Aspect 31: The device of Aspect 30, wherein each of the plurality of channels has a distal portion that is opposite from the central portion.

Aspect 32: The device of Aspect 31, wherein the channel is terminated with one or more inlets configured to be in fluid communication with the substrate.

Aspect 33: The device of Aspect 32, wherein the channel has two or more inlets fluidically connected to each other and to the channel.

Aspect 34: The device of Aspect 33, wherein the channel has at least three inlets fluidically connected to each other and to the channel.

Aspect 35: The device of any one of Aspects 32-34, wherein the substrate comprises a plurality of inlets that are substantially aligned with the one or more inlets of the plurality of channels, and wherein each of the plurality of inlets present on the substrate collects and transfer the sweat aliquot to each inlet of the plurality of channels.

Aspect 36: The device of Aspect 35, wherein the sweat aliquot collected by each inlet of the plurality of channels is transferred to the central portion of the at least one sweat collector element.

Aspect 37: The device of Aspect 35 or 36, wherein each of the plurality of inlets present on the substrate has a size that is the same or different and is effective to transfer the sweat aliquot to the one or more inlets of the plurality of channels by a capillary movement.

Aspect 38: The device of any one of Aspects 35-37, wherein the plurality of inlets present in the substrate are placed to ensure fluidic contact with the subject's skin.

Aspect 39: The device of any one of Aspects 1 or 30-38, further comprising a blocking layer disposed on at least a portion of the at least one sweat collector element, wherein the blocking layer comprises a plurality of apertures, wherein a first aperture of the plurality of apertures is aligned with the central portion of the at least one sweat collector element, and wherein remaining apertures are radially and outwardly disposed from the first aperture and aligned with at least a portion of each channel of the at least one sweat collector element to allow fluid communication with the at least one sweat collector element.

Aspect 40: The device of Aspect 39, further comprising a plurality of asymmetric membranes that are not in fluid communication with each other and are aligned with radial apertures of the blocking layer to allow fluid communication between asymmetric membranes and the at least one sweat collector element.

Aspect 41: The device of Aspect 40, wherein each of the plurality of asymmetric membranes is porous and has an axial gradient in pore size to allow a unidirectional axial flow of the sweat aliquot.

Aspect 42: The device of any one of Aspects 1 or 30-41, wherein the least one sensor portion comprises a plurality of sensing elements.

Aspect 43: The device of Aspect 42, wherein the plurality of sensing elements are not in fluid communication with each other.

Aspect 44: The device of Aspect 42 or 43, wherein the plurality of sensing elements are disposed above the plurality of asymmetric membranes and are in fluid communication with the plurality of asymmetric membranes.

Aspect 45: The device of any one of Aspects 42-44, wherein each of the plurality of sensing elements comprises a colorimetric reagent configured to react with at least one characteristic of the sweat.

Aspect 46: The device of Aspect 45, wherein the colorimetric reagent is present in an amount effective to determine a dynamic range of a concentration of the at least one characteristic of the sweat.

Aspect 47: The device of Aspect 44 or 45, wherein the colorimetric reagent is present in an effective composition to determine a dynamic range of a concentration of the at least one characteristic of the sweat.

Aspect 48: The device of Aspect 47, wherein the effective composition of the colorimetric reagent in each of the plurality of sensing elements is different and is configured to determine a different concentration of the at least one characteristic of the sweat.

Aspect 49: The device of any one of Aspects 39-48, further comprising an asymmetric indicator membrane having a bottom portion and an upper portion, wherein the bottom portion of the asymmetric indicator membrane is positioned above the first aperture and is in fluid communication with the at least one sweat collector element and is configured to transfer the sweat aliquot received from the first aperture axially.

Aspect 50: The device of Aspect 49, wherein the asymmetric indicator membrane is a porous membrane having an axial gradient in pore size, wherein the bottom portion of the asymmetric indicator membrane has a larger pore size than an upper portion, and wherein the upper portion of the asymmetric indicator membrane comprises a water-soluble dye configured to be dissolved in the sweat aliquot.

Aspect 51: The device of Aspect 49 or 50, wherein the asymmetric indicator membrane is positioned to receive the sweat aliquot after the colorimetric reagent present in each of the plurality of sensing elements reacted with the at least one characteristic of the sweat.

Aspect 52: The device of Aspect 51, wherein the asymmetric indicator membrane is configured to indicate that the at least one characteristic of the sweat is determined.

Aspect 53: The device of any one of Aspects 1 or 30-52, further comprising a masking layer.

Aspect 54: The device of Aspect 53, wherein the masking layer comprises a symbol, logo, or text that is configured to be visually observed when the at least one characteristic of the sweat is determined.

Aspect 55: The device of any one of Aspects 1 or 30-54, wherein the at least one characteristic of the sweat comprises an amount of cations, anions, pH, temperature, volume, an amount of biomarkers, or a combination thereof.

Aspect 56: The device of Aspect 53, wherein the at least one characteristic of the sweat is the amount of cations selected from sodium, potassium, calcium, magnesium, aluminum, iron, or a combination thereof.

Aspect 57: The device of Aspect 54, wherein the at least one characteristic of the sweat is the amount of anions selected from chlorides, lactates, or a combination thereof.

Aspect 58: The device of any one of Aspects 1 or 30-57, wherein the at least one characteristic of the sweat is present in an amount from about 0.10 mM to about 500 mM.

Aspect 59: The device of any one of Aspects 45-58, wherein the colorimetric reagent comprises silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion or iron ion, a 2,2-ticinchoninic acid, a, 10-phenanthroline, a universal pH indicator, silver dichromate, a compound of fluorescein and silver nitrate, or any combination thereof.

Aspect 60: The device of any one of Aspects 45-59, wherein the colorimetric reagent is impregnated within the at least one sensor portion from a solution, a suspension, or formed in-situ by reaction with the at least one characteristic of the sweat, or by a printing method.

Aspect 61: The device of any one of Aspects 1-60, wherein the device is disposable.

Aspect 62: A device comprising: a) a substrate that is configured to receive and transfer a fluid aliquot; b) at least one fluid collector element disposed on the substrate such that it is in fluid communication with the substrate and has at least one inlet configured to receive the fluid aliquot, wherein the at least one fluid collector element has a fluidic configuration such that a flow of the fluid aliquot is directed along a longitudinal axis and/or an axial axis of the at least one fluid collector element; c) at least one sensor portion that is in fluid communication with the at least one fluid collector element; wherein the at least one sensor portion is configured to quantitively detect at least one characteristic of the fluid; and wherein the device is configured to indicate a presence of the at least one characteristic of the fluid, wherein the fluid is a biofluid or a non-biofluid.

Aspect 63: The device of Aspect 62, wherein the biofluid comprises sweat, saliva, blood, urine, tears, reproductive fluids, or any combination thereof.

Aspect 64: The device of Aspect 62 or 63, wherein the non-biofluid comprises tap water, lake water, ocean water, wastewater, rain, water, groundwater, industrial process fluids, water from swimming pools, beverages, raw ingredients, or any combination thereof.

Aspect 65: A method comprising: a) providing the device of any one of Aspects 1-61; b) collecting the sweat aliquot within the device; and c) measuring at least one characteristic of the sweat within the at least one sensor portion of the device.

Aspect 66: A method comprising: a) providing the device of any one of Aspects 62-64; b) collecting the fluid aliquot within the device; and c) measuring at least one characteristic of the fluid within the at least one sensor portion of the device.

Aspect 67: A method of forming the device of any one of Aspects 1-64 comprising: a) providing a substrate configured to receive and transfer a fluid aliquot; b) disposing at least one fluid collector element such that is it in fluid communication with the substrate and has at least one inlet configured to receive the fluid aliquot; wherein the at least one fluid collector element has a fluidic configuration such that a flow of the fluid aliquot is directed along a longitudinal axis and/or an axial axis of the at least one fluid collector element; c) disposing at least one sensor portion that is in fluid communication with the at least one fluid collector element; wherein the at least one sensor portion is configured to quantitively detect at least one characteristic of the fluid; and wherein the device is configured to indicate a presence of the at least one characteristic of the fluid.

Aspect 68: The method of Aspect 67, wherein the at least one sweat collector element and/or the at least one sensor portion comprises an adsorbing material.

Aspect 69: The method of Aspect 67 or 68, wherein the at least one sensor portion comprises a colorimetric reagent configured to react with the at least one characteristic of the sweat.

Aspect 70: The method of Aspect 69, wherein the colorimetric reagent is impregnated with the at least one sensor portion of the device from a reagent solution.

Aspect 71: The method of Aspect 69, wherein the colorimetric reagent is deposited from a reagent suspension.

Aspect 72: The method of Aspect 69, wherein the colorimetric reagent is printed on the at least one sensor portion of the device.

Aspect 73: The method of Aspect 69, wherein the colorimetric reagent is formed in-situ.

Aspect 74: A device comprising: a) a substrate configured to be removably attached to a subject's skin, wherein the substrate has at least one inlet in fluid communication with at least the subject's skin and wherein the at least one inlet is configured to receive and transfer a sweat aliquot; b) at least one sweat collector element configured to receive the sweat aliquot from the at least one inlet of the substrate; c) at least one sensor element having a proximal end and a distal end, a first length, l₁, along a longitudinal axis of the device, measured between the proximal end and the distal end of the at least one sensor element, wherein the at least one sensor element is in fluid communication with the at least one sweat collector element; and wherein the at least one sensor element is configured to quantitatively detect at least one characteristic of the sweat; d) at least one ready indicator element that is in fluid communication with the at least one sweat collector element through at least one fluidic path having a proximal end and a distal end and having a second length, l₂, as measured between the proximal and distal ends along the longitudinal axis of the device, wherein the second length is greater than the first length (l₂>l₁); and wherein the device is configured to indicate a presence of the at least one characteristic of the sweat.

Aspect 75: The device of Aspect 74, wherein the at least one sweat collector element has a predetermined shape and a predetermined contact area.

Aspect 76: The device of Aspect 75, wherein the at least one inlet has a shape and a contact area that is the same or different as the predetermined shape and predetermined contact area of the at least one sweat collector element.

Aspect 77: The device of Aspect 75 or 76, wherein a portion of the at least one sweat collector element extends into the proximal end of the at least one fluidic path.

Aspect 78: The device of any one of Aspects 74-77, wherein the distal end of the at least one fluidic path extends into a portion of the at least one ready indicator element.

Aspect 79: The device of any one of Aspects 74-78, wherein the at least one ready indicator element has a predetermined shape having a predetermined area and is positioned substantially parallel to the at least one sweat collector element.

Aspect 80: The device of Aspect 79, wherein the predetermined shape and/or predetermined area of the at least one ready indicator element is the same or different from the predetermined shape and/or predetermined area of the at least one sweat collector element, respectively.

Aspect 81: The device of any one of Aspects 74-80, wherein the at least one fluidic path is configured to transfer a first portion of the collected sweat aliquot from the at least one sweat collector element to at least one ready indicator element.

Aspect 82: The device of any one of Aspects 74-81, wherein at least a portion of the at least one fluidic path comprises a dye configured to dissolve in the first portion of the sweat aliquot and be transferred to the ready indicator element.

Aspect 83: The device of any one of Aspects 74-82, wherein the at least one sensor element coupled to the at least one sweat collector element, such that it is positioned above the at least one fluidic path.

Aspect 84: The device of any one of Aspects 74-83, wherein the at least one sensor element is coupled to the at least one sweat collector element, such that it is positioned parallel to the at least one fluidic pat.

Aspect 85: The device of any one of Aspects 74-84, wherein the at least one sensor element comprises a reagent configured to react with the at least one characteristic of the sweat.

Aspect 86: The device of Aspect 85, wherein the reagent is a colorimetric reagent configured to change an original color of the reagent upon reaction with the at least one characteristic of the sweat.

Aspect 87: The device of Aspect 86, wherein the colorimetric reagent comprises silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion, or iron ion, a 2,2-ticinchoninic acid, a, 10-phenanthroline, a universal pH indicator, silver dichromate, a compound of fluorescein and silver nitrate, or any combination thereof.

Aspect 88: The device of any one of Aspects 85-87, wherein the reagent is impregnated within the at least one sensor element from a solution, a suspension, or formed in-situ by reaction with the at least one characteristic of the sweat, or by a printing method.

Aspect 89: The device of any one of Aspects 85-88, wherein the reagent is present in an amount effective to determine a dynamic range of a concentration of the at least one characteristic of the sweat.

Aspect 90: The device of any one of Aspects 85-89, wherein the reagent is present in an effective composition to determine a dynamic range of a concentration of the at least one characteristic of the sweat.

Aspect 91: The device of any one of Aspects 89-90, wherein the amount of the reagent is substantially similar per unit area.

Aspect 92: The device of any one of Aspects 74-91, wherein the at least one sensor element has a first width, d₁.

Aspect 93: The device of Aspect 92, wherein the first width, d₁, is the same or different along the first length, l₁.

Aspect 94: The device of any one of Aspects 74-93, wherein the at least one fluidic path has a second width, d₂.

Aspect 95: The device of Aspect 94, wherein the second width, d₂, is the same or different along the first length, l₂.

Aspect 96: The device of any one of Aspects 94-95, wherein the second width d₂ is the same as the first width d₁ (d₁=d₂) or wherein the second width d₂ is smaller than the first width d₁ (d₁>d₂).

Aspect 97: The device of any one of Aspects 83-96, wherein substantially no backflow is present between the at least one sensor element and the at least one fluidic path.

Aspect 98: The device of any one of Aspects 85-97, wherein the at least one characteristic of the sweat is exposed to an increasing cumulative amount of reagent as the sweat aliquot proceeds from the proximal end of the at least one sensor element to the distal end of the at least one sensor element.

Aspect 99: The device of Aspect 98, wherein the at least one sensor element is configured to indicate a concentration of the at least one characteristic of the sweat by a linear change of color of the colorimetric reagent from the proximal end to the distal end of the at least one sensor element.

Aspect 100: The device of any one of Aspects 74-99, wherein the at least one sweat collector element comprises a porous material configured to directionally adsorb and transfer the sweat aliquot.

Aspect 101: The device of Aspect 100, wherein the porous material comprises one or more layers.

Aspect 102: The device of any one of Aspects 74-101, wherein the at least one sweat collector element comprises a paper, a polymer, a textile, a foam, or any configuration thereof.

Aspect 103: The device of any one of Aspects 74-102, wherein the at least one characteristic of the sweat comprises an amount of cations, anions, pH, temperature, volume, an amount of biomarkers, or a combination thereof.

Aspect 104: The device of Aspect 103, wherein the at least one characteristic of the sweat is the amount of cations selected from sodium, potassium, calcium, magnesium, aluminum, iron, or a combination thereof.

Aspect 105: The device of Aspect 103 or 104, wherein the at least one characteristic of the sweat is the amount of anions selected from chlorides, lactates, or a combination thereof.

Aspect 106: The device of any one of Aspects 102-105, wherein the biomarkers comprise glucose, cholesterol, uric acid, urea, ascorbic acid, lactate, electrolytes, proinflammatory cytokines, hormones, tuberculosis-specific proteins, Parkinson-specific proteins, schizophrenia-specific proteins, cancer-specific metabolic markers, cystic fibrosis specific markers, drug-specific markers, alcohol, or any combination thereof.

Aspect 107: The device of any one of Aspects 74-106, wherein the at least one characteristic of the sweat is present in an amount from about 0.010 mM to about 500 mM.

Aspect 108: The device of any one of Aspects 74-107, wherein the substrate has a first surface facing the subject's skin and a second surface facing away from the subject's skin; and wherein the first surface of the substrate comprises an adhesive layer configured to removable attach the device to the subject's skin.

Aspect 109: The device of Aspect 108, wherein the adhesive layer comprises a polymer impermeable to the sweat.

Aspect 110: The device of Aspect 108 or 109, wherein the adhesive layer comprises an opening substantially aligned with the at least one inlet.

Aspect 111: The device of any one of Aspects 108-110, further comprising an opaque impermeable layer disposed between the adhesive layer and the first surface of the substrate.

Aspect 112: The device of Aspect 111, wherein the opaque impermeable layer has an opening substantially aligned with the at least one inlet.

Aspect 113: The device of any one of Aspects 108-112, wherein a peel-off layer is attached to the adhesive layer, wherein the peel-off layer is configured to protect the device when it is not in use and is configured to be removed in normal use.

Aspect 114: The device of any one of Aspects 74-113, wherein the at least one sweat collector element, at least one sensor element, at least one fluidic path, and/or at least one ready indicator element are disposed on the second surface of the device.

Aspect 115: The device of Aspect 114, further comprising an occlusive impermeable layer disposed above the at least one sweat collector element, at least one sensor element, at least one fluidic path, and/or at least one ready indicator element.

Aspect 116: The device of Aspect 115, further comprising a patterned upper surface configured to provide a visual indication of the presence of the at least one characteristic of the sweat and/or whether the device is ready for reading.

Aspect 117: The device of any one of Aspects 74-116, wherein the device further comprises an additional sensor element configured to sense a biological response different from one characteristic of the sweat.

Aspect 118: The device of Aspect 117, wherein the biological response comprises a heart rate, blood pressure, skin temperature, sweat rate, blood oxygen saturation, or any combination thereof.

Aspect 119: The device of any one of Aspects 74-118, wherein the device is disposable.

Aspect 120: The device of any one of Aspects 74-119, wherein the device is configured to be in communication with a processing unit.

Aspect 121: A device comprising: a) a substrate having at least one inlet that is configured to receive and transfer a fluid aliquot; b) at least one collector element configured to receive the fluid aliquot from the at least one inlet of the substrate; c) at least one sensor element having a proximal end and a distal end, a first length, l₁, along a longitudinal axis of the device, measured between the proximal end and the distal end of the at least one sensor element, wherein the sensor element is in fluid communication with the at least one collector; and wherein the at least one sensor element is configured to quantitatively detect at least one characteristic of the fluid; d) at least one ready indicator element that is in fluid communication with the at least one collector through at least one fluidic path having a proximal end and a distal end and having a second length, l₂, as measured between the proximal and distal ends along the longitudinal axis of the device, wherein the second length is greater than the first length (l₂>l₁); and wherein the device is configured to indicate a presence of the at least one characteristic of the fluid, wherein the fluid is a biofluid or a non-biofluid.

Aspect 122: The device of Aspect 121, wherein the biofluid comprises sweat, saliva, blood, urine, tears, reproductive fluids, or any combination thereof.

Aspect 123: The device of Aspect 122, wherein the non-biofluid comprises tap water, lake water, ocean water, wastewater, rain, water, groundwater, industrial process fluids, water from swimming pools, beverages, raw ingredients, or any combination thereof.

1. (canceled)

2. (canceled)

3. (canceled)

4. A device comprising:
a) a substrate configured to be removably attached to a subject's skin, wherein the substrate has at least one inlet in fluid communication with at least the subject's skin and wherein the at least one inlet is configured to receive and transfer a sweat aliquot;
b) at least one sweat collector element configured to receive the sweat aliquot from the at least one inlet of the substrate;
c) at least one sensor element having a proximal end and a distal end, a first length, l₁, along a longitudinal axis of the device, measured between the proximal end and the distal end of the at least one sensor element, wherein the at least one sensor element is in fluid communication with the at least one sweat collector element; and wherein the at least one sensor element is configured to quantitatively detect at least one characteristic of the sweat;
d) at least one ready indicator element that is in fluid communication with the at least one sweat collector element through at least one fluidic path having a proximal end and a distal end and having a second length, l₂, as measured between the proximal and distal ends along the longitudinal axis of the device, wherein the second length is greater than the first length (l₂>l₁); and
wherein the device is configured to indicate a presence of the at least one characteristic of the sweat.

5. The device of claim 4, wherein the at least one sweat collector element has a predetermined shape and a predetermined contact area, and wherein the at least one inlet has a shape and a contact area that is the same or different as the predetermined shape and predetermined contact area of the at least one sweat collector element.

6. (canceled)

7. The device of claim 5, wherein a portion of the at least one sweat collector element extends into the proximal end of the at least one fluidic path.

8. The device of claim 4, wherein the distal end of the at least one fluidic path extends into a portion of the at least one ready indicator element, and wherein the at least one ready indicator element has a predetermined shape having a predetermined area and is positioned substantially parallel to the at least one sweat collector element.

9. (canceled)

10. (canceled)

11. The device of claim 4, wherein the at least one fluidic path is configured to transfer a first portion of the collected sweat aliquot from the at least one sweat collector element to at least one ready indicator element.

12. The device of claim 4, wherein at least a portion of the at least one fluidic path comprises a dye configured to dissolve in the first portion of the sweat aliquot and be transferred to the ready indicator element.

13. The device of claim 4, wherein the at least one sensor element coupled to the at least one sweat collector element, such that it is positioned above the at least one fluidic path or wherein the at least one sensor element is coupled to the at least one sweat collector element, such that it is positioned parallel to the at least one fluidic path.

14. The device of claim 4, wherein the at least one sensor element comprises a reagent configured to react with the at least one characteristic of the sweat.

15. The device of claim 14, wherein the reagent is a colorimetric reagent configured to change an original color of the reagent upon reaction with the at least one characteristic of the sweat, and wherein the colorimetric reagent is present in an effective composition to determine a dynamic range of a concentration of the at least one characteristic of the sweat.

16. The device of claim 15, wherein the colorimetric reagent comprises silver chloroanilate, cobalt chloride, glucose oxidase, peroxidase, potassium iodide, lactate dehydrogenase, diaphorase, formazan dyes, 2,4,6-tris(2-pyridiyl)-s-triazine (TPTZ) complexed with mercury ion, silver ion, or iron ion, a 2,2-′bicinchoninic acid, a, 10-phenanthroline, a universal pH indicator, silver dichromate, a compound of fluorescein and silver nitrate, or any combination thereof.

17. The device of claim 14, wherein the reagent is impregnated within the at least one sensor element from a solution, a suspension, or formed in-situ by reaction with the at least one characteristic of the sweat, or by a printing method.

18. The device of claim 4, wherein the at least one sweat collector element comprises a porous material configured to directionally adsorb and transfer the sweat aliquot.

19. The device of claim 4, wherein the at least one sweat collector element comprises a paper, a polymer, a textile, a foam, or any configuration thereof.

20. The device of claim 4, wherein the at least one characteristic of the sweat comprises an amount of cations, anions, pH, temperature, volume, an amount of biomarkers, or a combination thereof.

21. The device of claim 20, wherein the at least one characteristic of the sweat is:
a) the amount of cations selected from sodium, potassium, calcium, magnesium, aluminum, iron, or a combination thereof, or
b) the amount of anions selected from chlorides, lactates, or a combination thereof; and/or
c) wherein the biomarkers comprise glucose, cholesterol, uric acid, urea, ascorbic acid, lactate, electrolytes, proinflammatory cytokines, hormones, tuberculosis-specific proteins, Parkinson-specific proteins, schizophrenia-specific proteins, cancer-specific metabolic markers, cystic fibrosis specific markers, drug-specific markers, alcohol, or any combination thereof.

22. The device of claim 4, wherein the at least one characteristic of the sweat is present in an amount from about 0.10 mM to about 500 mM.

23. The device of claim 4, wherein the device further comprises an additional sensor element configured to sense a biological response different from one characteristic of the sweat and wherein the biological response comprises a heart rate, blood pressure, skin temperature, sweat rate, blood oxygen saturation, or any combination thereof.

24. A device comprising:
a) a substrate that is configured to receive and transfer a fluid aliquot;
b) at least one fluid collector element disposed on the substrate such that it is in fluid communication with the substrate and has at least one inlet configured to receive the fluid aliquot;
wherein the at least one fluid collector element has a fluidic configuration such that a flow of the fluid aliquot is directed along a longitudinal axis and/or an axial axis of the at least one fluid collector element;
c) at least one sensor portion that is in fluid communication with the at least one fluid collector element; wherein the at least one sensor portion is configured to quantitively detect at least one characteristic of the fluid; and
wherein the device is configured to indicate an amount of the at least one characteristic of the fluid, wherein the fluid is a biofluid or a non-biofluid.

25. A device comprising:
a) a substrate having at least one inlet that is configured to receive and transfer a fluid aliquot;
b) at least one collector element configured to receive the fluid aliquot from the at least one inlet of the substrate;
c) at least one sensor element having a proximal end and a distal end, a first length, l₁, along a longitudinal axis of the device, measured between the proximal end and the distal end of the at least one sensor element, wherein the sensor element is in fluid communication with the at least one collector; and wherein the at least one sensor element is configured to quantitatively detect at least one characteristic of the fluid;
d) at least one ready indicator element that is in fluid communication with the at least one collector through at least one fluidic path having a proximal end and a distal end and having a second length, l₂, as measured between the proximal and distal ends along the longitudinal axis of the device, wherein the second length is greater than the first length (l₂>l₁); and
wherein the device is configured to indicate a presence of the at least one characteristic of the fluid, wherein the fluid is a biofluid or a non-biofluid.

26. (canceled)
